SEQUENCE LISTING (6 sequences)

SEQ ID NO: 1 - DNA, 22 bp, Artificial Sequence
  Primer
ttgttcttctgcttgtgtccct

SEQ ID NO: 2 - DNA, 22 bp, Artificial Sequence
  Primer
agtcgaggcagatgccatagtt

SEQ ID NO: 3 - DNA, 20 bp, Artificial Sequence
  Primer
aagcccatcaccatcttcca

SEQ ID NO: 4 - DNA, 20 bp, Artificial Sequence
  Primer
atggcatggactgtggtcat

SEQ ID NO: 5 - DNA, 24 bp, Artificial Sequence
  Primer
cggttggacccatcggatgccatg

SEQ ID NO: 6 - DNA, 24 bp, Artificial Sequence
  Primer
gaactctttcccgtctttaccgcg

(19) United States
(12) Patent Application Publication
(10) Pub. No.: US 2007/0213295 A1
(43) Pub. Date: Sep. 13, 2007

(54) METHODS AND COMPOSITIONS FOR INDUCING TORPOR IN A SUBJECT

(75) Inventors: Cheng Chi Lee, Houston, TX (US); Jianfa Zhang, Houston, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/623,625

(22) Filed: Jan. 16, 2007

Related U.S. Application Data
(60) Provisional application No. 60/821,521, filed on Aug. 4, 2006.
(60) Provisional application No. 60/759,480, filed on Jan. 16, 2006.

(51) Int. Cl. A61K 31/7076

(52) U.S. Cl. 514/47

(57) ABSTRACT

The present invention relates to the discovery the 5′-AMP and analogues thereof can be used to induce a state of torpor or suspended animation in subjects, as exemplified by studies carried out in laboratory mice. In these studies, mice were injected with high doses of 5′-AMP, which was found to result in a decoupling of the animals' body temperature regulation mechanism accompanied by a reduction in the animals' core body temperature, which tended to lower towards the ambient environmental temperature. It was further discovered that the introduction of high levels of 5′-AMP resulted in an induction of fat regulation genes such as procolipase (Clps) in tissues and organs that do not normally express Clps, this in turn was accompanied by a shift in metabolism from a primarily glycolytic energy metabolism (which is inhibited at lower temperatures) to one that relied primarily on the liberation and metabolism of free fatty acids. Substantial medical and other applications that arise out of this discovery are also disclosed.

The present application claims the benefit of U.S. provisional application Ser. No. 60/759,480, filed Jan. 16, 2006 and Ser. No. 60/821,521, filed Aug. 4, 2006, the disclosures of which are incorporated herein by reference.

The U.S. Government owns rights in this invention pursuant to NIH grant 1 R01 AG 20912-01A1.

### BACKGROUND OF THE INVENTION

1. Field of the Invention

The present invention relates in general to inducing a state of torpor or suspended animation, and more particularly, to compositions and methods of using 5′adenosine monophosphate (“5′-AMP”), or an analogue thereof, to modulate the core body temperature and metabolic rate of a subject.

2. Description of the Related Art

Hibernation, effected by a state of torpor or suspended animation (a severe hypothermic state), is the mechanism use by animals to adapt to the environment and surroundings, allowing animals to live in harsh climates with challenging metabolic need. Hibernation or torpor allows the animal to survive the winter conditions by lower their metabolism during times of cold temperatures and scarcity of food, e.g., during the winter.

Generally, hibernation allows the body to utilize stored body fat instead of glucose as the major energy source. Reduction in body movement and the core body temperature are the major mechanisms used to reduce energy consumption by the animal. The core body temperature (“CBT”) of the animal can be lowered significantly to several degrees above ambient room temperature, e.g., from 98.6 degrees Fahrenheit to as low as several degrees above 32 degrees Fahrenheit (e.g., in the alpine marmot, Siberian hamster or in ground squirrels). This allows energy that is normally used to maintain the body temperature to be diverted to other needs. In addition to a reduction in the body temperature, the heart rate and breathing rate of the animal also decreases. A change in energy source from primary glucose to more free (e.g., non-esterified) fatty acids has been demonstrated in the metabolism of the animal during hibernation.

Metabolic-related issues like obesity represents a growing major public health problem in the Unites States and across the industrial world. Currently, about 60% of the adult population is regarded as obese in the United States and morbid obesity is observed in about 30% of the adult population. Obesity has been associated with many diseases including insulin-resistant type II diabetes, hypertension, hyperlipidemia, heart attacks and increased mortality rate. Obesity is the result of a positive energy balance due to an excess in the caloric intake relative to the energy expenditure, which is consequentially stored by the body.

There are various proposed mechanisms related to obesity. For example, studies have found that mutations in certain genes (e.g., ob, fa/fa and db) lead to a complex, clinically similar phenotype of obesity. These phenotypes demonstrate characteristics evident starting at about one month of age, which includes hyperphagia, severe abnormalities in glucose and insulin metabolism, very poor thermoregulation and non-shivering thermogenesis, and extreme torpor and underdevelopment of the lean body mass. From these studies, torpor also seems to play a larger part in the maintenance of obesity and temperature, e.g., inbred mouse strains such as NZO mice and Japanese KK mice that are moderately obese. Certain hybrid mice, such as the Wellesley mouse, become spontaneously fat. Furthermore, desert rodents, e.g., the spiny mouse, become obese when fed with standard laboratory feed. Therefore a well-tolerated agent that effectively and simultaneously treats the factors associated with the obesity would have a significant impact on the prevention and treatment of diseases associated with obesity, e.g., hypertension, diabetes, heart attacks and atherosclerosis.

There are also reports of proposed approaches to addressing the foregoing needs. For example, U.S. Pat. No. 6,979,750, issued to Scanlan, et al., teaches thyronamine derivatives and analogs, that are said to exert a positive inotropic effect on the heart without affecting the heart rate, lower the core body temperature and induce states of torpor or hibernation. The patent relates to thyronamine derivatives and analogs of thyroid hormone. Thyroid hormone is an important regulator of vertebrate development and homeostasis. In adults, thyroid hormone exerts effects in almost all tissues, and important processes such as metabolic rate, thermal regulation, lipid inventory, cardiac function, and bone maintenance are affected by the thyroid hormone. Individuals with excess blood levels of the thyroid hormone (hyperthyroid) generally have elevated metabolic rate and body temperature, decreased serum cholesterol, and increased heart rate compared to those with normal thyroid hormone levels (e.g., euthyroid). Conversely, hypothyroidism is characterized by a depressed metabolic rate and body temperature, elevated serum cholesterol, and decreased heart rate compared to euthyroid controls.

U.S. Patent Application 2005/0136125 by Roth is directed towards the use of oxygen antagonists to induce stasis in a tissue or organism. The oxygen antagonist reduces the amount of oxygen available to the tissue or organism, and in some embodiments, an inhibitor of cytochrome c oxidase (e.g., carbon monoxide or hydrogen sulfide) may be used. The well-known toxicities of many oxygen antagonists may, in certain instances, present a disadvantage of this approach. For example, oxygen antagonists such as hydrogen cyanide, which has been used as a chemical weapon, possess toxicities that may limit their use in many settings.

The above approaches suffer from significant drawbacks in that the compounds employed may have unintended or undesirable secondary effects, such as untoward effects due to their hormonal activity. These approaches often use compound(s) not produced naturally or used physiologically by a mammal. Thus, there exists a substantial need for compositions, and associated methods, that can be employed to induce states of torpor or suspended animation in subjects, including both man and laboratory animals that, e.g., have the ability to reduce the metabolic activity of the subject, including mediators of fatty acid utilization, and/or safely permit reductions in body temperature during times of need.

### SUMMARY OF THE INVENTION

The present inventors recognized a need for the control of metabolic pathways by inducing a state of torpor or suspended animation in a subject to modulate the core body temperature and metabolic rate, as an example. Generally, hibernation or torpor is used by animals to conserve energy during episodes of food or metabolic stress such as winter. In mammals, the circadian clock has been implicated in this role since there is an association between daily torpor (e.g., a short hibernation-like state) and the body temperature rhythm. Additionally, the photoperiod length regulates daily torpor rhythm and body weight of mammals. Classic hibernation is only observed in rodents such as ground squirrels and large mammals such as bears. Laboratory mice, like humans, cannot hibernate; however, mice can undergo shallow torpor during metabolic stress such as fasting indicating that some basic mechanisms for hibernation are retained. As used herein, “torpor” is defined as a state of extreme lethargy or loss of wakefulness, associated with a loss of the body's normal body temperature regulation, hence, leading to a migration of the body temperature towards the surrounding, environmental ambient temperature. Typically, an animal (e.g., a mouse) is said to be in a state of torpor when the animal's core body temperature is lowered to at least about 31° C. or less. As used herein, “hypothermia” is defined as having a core body temperature which is colder than the physiological norm for that organism; typically, a mammal is in a state of hypothermia when the mammal's core body temperature is reduced below about 37° C. In certain embodiments, hypothermia may be induced in a human to lower the core body temperature to from about 15° C. to about 36° C., more preferably from about 17° C. to about 35° C., more preferably from about 25° C. to about 34° C., and in certain embodiments, the core body temperature of the human may be lowered to from about 27° C. to about 33° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or any temperature derivable therein. A human is said to be in “severe hypothermia” when the core body temperature of the human drops to about 28° C. or below.

The present invention arose in part out of the inventors' recognition that mice kept in constant darkness have inverted metabolic fundamentals, similar to that observed in hibernating mammals. For example, most warm-blooded animals, such as mammals, rely primarily on a glycolytic metabolism in oxidative phosphorylation and glycolysis for their energy needs, using glucose as a preferred energy source. However, the present inventors observed that mice maintained in a state of constant darkness shifted their metabolism to rely more on fatty acid metabolism, rather than glucose metabolism. Indeed, it was found that their blood glucose and fatty acids levels are reverse to that of mice kept in regular light-dark cycles. In addition, these animals eat less and lose body weight compared to mice kept in normal light-dark cycle.

The present inventors discovered that genes encoding for the enzyme(s) controlling fat breakdown, particularly procolipase (Clps) (and its enzymatic partners pancreatic lipase related protein 2 (plrp2), pancreatic lipase related protein 1 (plrp1) and likely pancreatic triacylglycerol lipase (PTL)) was activated in peripheral organs when mice were kept in constant darkness but not in the light-dark cycle. Until the present invention, it has only been shown that these enzymes are expressed in pancreas and gastrointestinal organs consistent with their role in dietary fat degradation. Additionally the present inventors recognized that the circadian clock plays a major role in mediating this molecular response. A consequence of the foregoing observation, and a key aspect of the present invention, was the discovery that 5′-adenosine monophosphate (5′-AMP), was a key mediator of this signaling mechanism.

It was, for example, recognized that mice kept in constant darkness (“DD”) had increased level of blood 5′-AMP relative to mice maintained in a conventional light-dark (“LD”) cycle. When 5-AMP was administered to mice in high amounts, the mice went into a state of torpor, as evidenced by a state of extreme lethargy and loss of wakefulness accompanied by a reduced core body temperature (CBT) relative to normal CBT. The metabolic changes mediated by 5′-AMP and constant darkness are likely an evolutionary mechanism used by mammals to conserve energy. For example, mice undergo torpor in a response to metabolic stress. For example, mice undergo torpor in a response to metabolic stress, e.g., during short fasting periods (e.g., between 2-3 days) in constant darkness but not in light-dark cycle. Factors such as the size of the animal and the environmental temperature can also influence the onset of torpor; for example, smaller, leaner animals and colder environmental temperatures can accelerate the onset or presence of torpor. In addition, as a response to such metabolic stress the blood 5′-AMP level increases dramatically, demonstrating that physiologically regulated 5′-AMP is associated with or is a mediator of the torpor response. When synthetic 5′-AMP was injected into mice the level of glucose was regulated and was reciprocally linked with the expression of the procolipase gene.

The present inventors recognized 5′-AMP as a pivotal switch that regulate the energy balance in mammals between glucose, glycogen and fat. 5′-AMP is an allosteric regulator of several rate-limiting enzymes controlling glycolysis (Phosphofructose kinase (PFK) and gluconeogenesis (Fructose 1,6-diphosphatase (FDP, and Glycogen breakdown (Glycogen Phosphorylase (subunits alpha and beta)). The present inventors also identified fat catabolism genes (e.g., procolipase (CLPS) and its enzymatic partners pancreatic lipase related protein 2 (plrp2), pancreatic lipase related protein 1 (plrp1) and likely pancreatic triacylglycerol lipase (PTL)) as the metabolic mechanism that is under circadian regulation in mammals by virtue of 5′-AMP action.

An aspect of the present invention relates to a method of inducing a state of hypothermia, torpor, or suspended animation in a subject comprising administering an amount of 5′-AMP, or a non-naturally occurring, synthetic analogue of 5′-AMP (e.g., wherein the analogue induces mClps, PTL, PLRP1 or PLRP2), to the subject that is effective to induce the state of torpor or suspended animation. The method may further comprising determining that the subject is in a state of torpor or suspended animation. In certain embodiments, the method further comprises subjecting the subject to an ambient environmental temperature that is below about 30° C. (e.g., between about 25° C. and about 1° C.; or between about 20° C. and about 4° C.) after administration of the 5′-AMP or analogue.

In certain embodiments, steps may be taken to alter the rate at which the body temperature of the subject changes. For example, in certain embodiments, the ambient environment may comprise a water bath, wherein the subject is at least partially submerged in the water bath. Altering the room temperature can also provide a means to affect the rate at which the body temperature of the subject changes. It is envisioned that other methods for altering the rate of hypothermia may also be used with the present invention.

The method may further comprises lowering the core body temperature of the subject below about 37° C. (e.g., about 32° C. or less, between about 15° C. and about 20° C., or between about 13° C. and about 15° C.). In certain embodiments, the torpor is deep torpor or a severe hypothermic state.

The 5′-AMP or analogue may be administered by subcutaneous injection, intramuscular injection, intravenous injection, intraperitoneal injection, nasal administration, intravaginal administration, intranasal administration, intrabronchial administration, intraocular administration, intraaural administration, intracranial administration, oral consumption, parenteral administration, rectal administration, sublingual administration, topical administration, transdermal administration or combination thereof. The 5′-AMP or analogue may be administered in the form of, for example, a capsule, caplet, softgel, gelcap, suppository, film, granule, gum, pastille, pellet, chewable tablet, troche, lozenge, disk, poultice, wafer, creams, lotions, ointments, aerosol sprays, roll-on liquids, roll-on sticks, transdermal patches, subcutaneous implants, pads or combinations thereof. In certain embodiments, the 5′-AMP or analogue is disposed for extended release in a biodegradable carrier.

The 5′-AMP or analogue may be administered in a pharmaceutically acceptable dosage form that further comprises a pharmaceutically acceptable carrier. In certain embodiments, the pharmaceutically acceptable carrier comprises an aerosol propellant selected from nitrogen, carbon dioxide, propane, butane, isobutene, pentane, isopropane, fluorocarbons, dimethylether and mixtures thereof. The 5′-AMP or analogue may be administered in a dosage form that further comprises at least one agent selected from the group consisting of emollients, water, inorganic powders, foaming agents, emulsifiers, fatty alcohols, fatty acids and combinations thereof.

In certain embodiments, the subject is a human or a laboratory animal (e.g., a mouse). The effective amount of 5′-AMP or analogue may range from about 5 mg/kg to about 7.5 gm/kg body weight, from about 15 mg/kg to about 1.5 gm/kg body weight, from about 5 mg/kg to about 15 mg/kg, or from about 25 mg/kg to about 250 mg/kg body weight. In embodiments where the effective amount ranges from about 5 mg/kg to about 15 mg/kg, the method may further comprise rapidly lowering the body temperature of the subject (e.g., by using a water bath) in order to induce a state of torpor. The method may further comprise at least partially submerging the subject in a water bath, wherein the temperature of the water bath is below about 32° C. The method may further comprises at least partially covering the patient with a cooling blanket.

For example, the inventors have determined that, in a mouse, the 5′-AMP ED₅₀ for inducing torpor using a 4° C. environment for the cooling phase (subsequent body temperature can be maintained at the desired temperature, e.g., 4° C. to 28° C.) is approximately 0.15 mg/gram body weight, and the 5′-AMP ED₁₀₀ is approximately 0.25 mg/g body weight. The inventors have also determined that a dose of about 5-7.5 mg/g body weight of 5′-AMP can induce deep torpor in a mouse.

The subject may be suffering from a disease state to be treated by induction of a state of torpor or suspended animation. For example, the disease state may be a state of shock, trauma, a blood coagulation disorder, a side effect of chemotherapy, poisoning, a cardiac arrhythmia, hypothermia, burns, suffocation, inhalation injury, ventilation insufficiency, sepsis, anxiety, insulin shock, an infectious disease, cancer, carcinoma, near drowning, heart attack, congestive heart failure, decompression sickness, asthma, starvation, stroke, severe trauma, a head trauma, a brain trauma, a cerebrovascular injury, a cerebrovascular trauma, a neurological trauma, a neurological injury, a fever, a heatstroke, an eating disorder, anxiety, a seizure, epilepsy, insomnia or a sleeping disorder, diabetes, obesity, hypertension, hyperthyroidism, hypothyroidism or combinations thereof. The subject may be a transplant recipient, a transplant donor, in need of appetite suppression, a pre-surgical patient, a post-surgical patient, or a patient who has received or will be receiving a chemotherapeutic.

The amount of 5′-AMP or analogue may be effective to reduce the core body temperature, reduce the external body temperature, reduce the metabolic rate, reduce the heart rate and combinations thereof. The method may further comprise administering to the subject a second pharmaceutical agent. The second pharmaceutical agent may comprise an adjunctive agent, heparin, an anticoagulant, an inotropic agent, a chronotropic agent, an analgesic agent, an anesthetic agent, a neuroprotective agent, an antiarrhythmic agent, or a calcium channel blocker.

Another aspect of the present invention involves a method of reducing blood glucose level in a subject comprising administering to the subject an amount of 5′-AMP, or a non-naturally occurring, synthetic analogue of 5′-AMP that induces mClps, PTL, PLRP1 or PLRP2, effective to reduce blood glucose levels in the individual. The subject may be suffering from diabetes, obesity or is in need of appetite suppression.

Another aspect of the present invention involves a method of modifying the metabolic state of a tissue to increase fatty acid metabolism in the tissue relative to glycolysis therein, comprising administering to the subject an amount of 5′-AMP, or a non-naturally occurring, synthetic analogue of 5′-AMP that induces mClps, PTL, PLRP1 or PLRP2, effective to increase fatty acid metabolism. The tissue may be comprised in a subject. The subject may be a transplant recipient, a transplant donor, a pre-surgical patient, a post-surgical patient, or a patient who has received or will be receiving a chemotherapeutic. In certain embodiments, the tissue has been removed from a subject. The tissue may comprise part or all of an organ (e.g., a transplant organ). The method may further comprise determining that the fatty acid metabolism in the subject has been increased. In certain embodiments, the patient is suffering from diabetes, obesity or is in need of appetite suppression. In certain embodiments, the tissue is a solid tumor.

Another aspect of the present invention involves a method of reducing the core body temperature of a subject comprising administering to the subject an amount of 5′-AMP, or a non-naturally occurring, synthetic analogue of 5′-AMP that induces mClps, PTL, PLRP1 or PLRP2, that is effective to reduce the subject's core body temperature. The method may further comprise determining the subject's core body temperature. The subject may be in a state of shock, have a trauma, a blood coagulation disorder, side effects of chemotherapy, poisoning, a cardiac arrhythmia, hypothermia, burns, suffocation, an inhalation injury, ventilation insufficiency, sepsis, anxiety, insulin shock, an infectious disease, cancer, carcinoma, near drowning, heart attack, congestive heart failure, decompression sickness, asthma, starvation, in need of appetite suppression, stroke, severe trauma, a head trauma, a brain trauma, a cerebrovascular injury, a cerebrovascular trauma, a neurological trauma, a neurological injury, a fever, a heatstroke, an eating disorder, anxiety, a seizure, epilepsy, insomnia and sleeping disorders, diabetes, obesity, hypertension, hyperthyroidism, hypothyroidism, is to undergo a surgical procedure, is a transplant patient, is a patient who has received or will be receiving a chemotherapeutic, or combinations thereof.

Another aspect of the present invention relates to a method of reducing the metabolic rate of a subject comprising administering to the subject an amount of 5′-AMP, or a non-naturally occurring, synthetic analogue of 5′-AMP that induces mClps, PTL, PLRP1 or PLRP2, that is effective to reduce the subject's metabolic rate. The method may further comprise assessing the subject's metabolic rate. The subject may be in a state of shock, have a trauma, a blood coagulation disorder, side effects of chemotherapy, poisoning, a cardiac arrhythmia, hypothermia, burns, suffocation, inhalation injury, ventilation insufficiency, sepsis, anxiety, insulin shock, an infectious disease, cancer, carcinoma, near drowning, heart attack, congestive heart failure, decompression sickness, asthma, starvation, stroke, severe trauma, a head trauma, a brain trauma, a cerebrovascular injury, a cerebrovascular trauma, a neurological trauma, a neurological injury, a fever, a heatstroke, an eating disorder, anxiety, a seizure, epilepsy, insomnia and sleeping disorders, diabetes, obesity, hypertension, hyperthyroidism, hypothyroidism, is to undergo a surgical procedure, is a transplant patient, is in need of appetite suppression, is a patient who has received or will be receiving a chemotherapeutic, or combinations thereof.

Another aspect of the present invention relates to a pharmaceutical composition comprising a pharmaceutically effective amount of 5′-AMP, or a non-naturally occurring, synthetic analogue of 5′-AMP that induces mClps, PTL, PLRP1 or PLRP2, sufficient to produce a state of torpor or suspended animation, a reduction in the core body temperature, an inotropic effect on the heart, a decrease in cell growth, a reduction in metabolic rate, a reduction in the blood glucose levels or combinations thereof. The composition may be formulated for administration by subcutaneous injection, intramuscular injection, intravenous injection, intraperitoneal injection, nasal administration, intravaginal administration, intranasal administration, intrabronchial administration, intraocular administration, intraaural administration, intracranial administration, oral consumption, parenteral administration, rectal administration, sublingual administration, topical administration, transdermal administration or combination thereof. The 5′-AMP or analogue may be formulated in the form of a capsule, caplet, softgel, gelcap, suppository, film, granule, gum, insert, a chewable tablet, a pastille, pellet, troche, lozenge, disk, poultice, wafer, a cream, a lotion, ointments, aerosol sprays, roll-on liquids, roll-on sticks, transdermal patches, subcutaneous implants, pads, or is disposed for extended release in a biodegradable carrier.

The pharmaceutical composition may further comprising a pharmaceutically acceptable carrier. The pharmaceutically acceptable carrier may comprise an aerosol propellant selected from nitrogen, carbon dioxide, propane, butane, isobutene, pentane, isopropane, fluorocarbons, dimethylether and mixtures thereof. The pharmaceutically acceptable carrier may comprise at least one agent selected from the group consisting of emollients, water, inorganic powders, foaming agents, emulsifiers, fatty alcohols, fatty acids and combinations thereof. The pharmaceutical composition may be formulated in an injectable dosage form. The 5′-AMP or analogue may be contained in a metered dosage in a vial or ampoule. In certain embodiments, the 5′-AMP or analogue is dispersed in an aqueous solution.

In certain embodiments, the pharmaceutical composition further comprises one or more pharmaceutical additives. The additives may include one or more buffers or physiologic salts. The vial or ampoule may comprise a septum. The dosage may be metered to provide from 1 to 5 doses. Each dose may comprise from 1 to 500 gm, or from 2 to 20 gm of 5′-AMP or analogue. In certain embodiments, each dose comprises an amount of 5′-AMP or analogue effective to deliver from 15 mg/kg to 7.5 gm/kg body weight, or from 25 mg/kg to 250 mg/kg body weight, to a subject. The pharmaceutical composition may be formulated and placed into a projectile for inducing a state of torpor or suspended animation in a subject. In certain embodiments, the pharmaceutical composition is further defined as an aerosol composition adapted for inducing torpor in a subject comprising a pharmaceutically effective amount of 5′-AMP and a propellant. The aerosol composition may be in the form of an inhaler.

In certain embodiments, the pharmaceutical composition further comprises a second active agent. The second active agent may be an adjunctive agent, heparin, an anticoagulant, an inotropic agent, a chronotropic agent, an analgesic agent, an anesthetic agent, a neuroprotective agent, an antiarrhythmic agent, or a calcium channel blocker.

Another aspect of the present invention relates to a method of altering metabolic activity in a subject comprising administering a pharmaceutically effective amount of 5′-AMP to a subject, wherein the 5′-AMP or a precursor of 5′-AMP alters the activity of one or more metabolic enzymes selected from procolipase, pancreatic lipase, pancreatic lipase related protein, phosphofructose kinase, fructose 1,6 diphosphatase, glycogen phosphorylase, and combinations thereof.

Another aspect of the present invention relates to a transport vehicular anti-terrorism security system for inducing torpor in one or more subjects on the vehicle comprising: a crew compartment, a passenger cabin; a pressurized security system disposed within the passenger cabin comprising one or more outlets in the cabin, wherein the pressurized security system comprises a pharmaceutically effective amount of 5′-AMP or a precursor of 5′-AMP and a propellant; one or more activation mechanisms, wherein the activation of the one or more activation mechanisms results in the release of the 5′-AMP or a precursor of 5′-AMP induces into the crew compartment, the passenger cabin or both and induces torpor when inhaled by the one or more subjects; and an isolated air system in the crew cabin for one or more pilots. The one or more outlets may be configured to interface with the vehicle air circulation system. The isolated air system may comprise one or more masks for the one or more pilots, a sealed cockpit crew cabin or combinations thereof. In certain embodiments, the vehicle is an aircraft, a ground vehicle, or a water craft.

### BRIEF DESCRIPTION OF THE DRAWINGS

For a more complete understanding of the features and advantages of the present invention, reference is now made to the detailed description of the invention along with the accompanying figures and in which:

FIGS. 1a and 1b are images of a cDNA micro-array;

FIG. 2 is an image of an autoradiogram that illustrates in Clps expression in liver of light-dark mice;

FIG. 3 is an image of an autoradiogram blot that illustrates mClps expression in liver of constant darkness mice;

FIGS. 4a and 4b are images of autoradiograms that illustrate mClps expression in the liver and adipose tissue respectively;

FIG. 5 is a graph illustrating the hydrolysis of triacylglycerol analog by liver protein per time;

FIG. 6 is an image of an autoradiogram that illustrates the expression of mClps in various peripheral tissues and brain tissue;

FIG. 7 is an image of an autoradiogram that reveals that both mClps and mPlrp2 expression;

FIG. 8 is a chromatogram illustrating the retention times of various peaks analyzed by reverse phase HPLC in light-dark and constant darkness mice;

FIG. 9 is a plot of absorbance verses time that examines the diurnal pattern of various HPLC peaks;

FIG. 10 is a bar chart of the absorbance over time to display differential level for constant darkness mice and light-dark mice;

FIG. 11 is a graph comparing the retention time of various samples and compounds which identify peak 2 to be 5′-AMP;

FIG. 12 is an image of an autoradiogram that demonstrates 5′-AMP at various concentrations induced mClps expression in the liver;

FIG. 13 is an image of an autoradiogram that illustrates the induction of mClps expression at about 3.5 to 4 hours after the 5′-AMP was injected in relation with the blood glucose level FIG. 28;

FIG. 14 is an image of a gel that illustrates 5-AMP induction of mClps expression in all peripheral tissues sampled except the brain using reverse transcriptase polymerase chain reaction techniques;

FIG. 15 is an image of a Northern blot examining the intracellular action of 5′-AMP via adenosine receptors or transporters;

FIG. 16 is an image of an autoradiogram examining the blocking of adenosine and 5′-AMP induced colipase induction by dipyridamole, a potent inhibitor of nucleoside transporters;

FIG. 17 is an image of an autoradiogram analyzing adenine nucleotides induction of mClps expression in the liver;

FIG. 18 is a graph of temperature verses time after administering 5′-AMP;

FIG. 19 is a graph of temperature verses time after administering 5′-AMP;

FIG. 20 is a graph of temperature verses time after administering 5′-AMP to examine the effect of metabolic stress;

FIG. 21 is a HPLC chromatogram comparing 5′-AMP peak sizes during a torpor state and a non-torpor state;

FIG. 22 is a bar graph that quantifies the relative HPLC peak sizes of 5′-AMP;

FIGS. 23a-23c are plots demonstrating physiological control of 5′-AMP levels and induction of torpor as a result of metabolic stress; FIG. 23a is a graph of temperature verses time after administering 5′-AMP; FIG. 23b is a HPLC chromatogram comparing 5′-AMP peak sizes during a torpor state and a non-torpor state; and FIG. 23c is a bar graph that quantifies the relative HPLC peak sizes revealed that 5′-AMP levels;

FIGS. 24a and 24b are graphs of consumption of food and water per day;

FIG. 25 is a graph of body weight per day;

FIG. 26 is a graph of free fatty acids in the serum over time;

FIG. 27 is a graph of glucose concentration over time:

FIG. 28 is a graph of the concentration of glucose in the blood over time;

FIG. 29 is an image of an autoradiogram illustrating the 5′-AMP activation of mClps expression in light-dark mice;

FIG. 30 is a chart illustrating the role of 5%-AMP in metabolic signaling;

FIGS. 31a and 31b are graphs of the food intake and body weight of morbid obese mice with daily injection of 5′-AMP;

FIGS. 32a and 32b are graphs of the food intake and body weight of morbid obese mice kept in constant darkness and kept in regular light-dark;

FIG. 33 is an image of a Northern blot illustrating the expression of ecto-5′nucleotidase gene is high in light-dark cycle mice but low in mice kept in constant darkness;

FIGS. 34 a and b show the entry into and length of SA as a function of 5′-AMP concentration. FIG. 34 a: Titration of 5′-AMP dose as a function of its ability to induce mice (n=4) to enter SA at 4° C. AET in 60 min. FIG. 34 b: The length of SA as a function of the injected concentration of 5′-AMP (milligrams per gram body weight (mg/gbw)). Once animals entered SA, they were maintained at 15±0.5° C. until spontaneous arousal was observed. Arousal was defined as the ability of the mouse to undertake RF spontaneously;

FIGS. 35 a, b and c show the effect of AET on CBT and length of SA in mice. FIG. 35 a: The CBT of individual animals kept at 14° C. or 15° C. AET after 2 h in SA. FIG. 35 b: The length of SA of mice maintained at 14° C. or 15° C. AET. Note: animals in SA were rescued after 12 h by transferring them to a 20-22° C. ambient laboratory temperatures while awaiting for spontaneous arousal. FIG. 35 c: Arousal from SA and the rise in CBT of mice maintained at various AET. Each group of mice (n=4) were put into SA and then transferred into incubators at the indicated temperature to observe the recovery rate of CBT. SA in these studies was carried out with 0.5 mg/gbw of 5′-AMP;

FIG. 36 shows a proposed model to explain the interacting role between 5′-AMP and hypothermia during SA. Model showing the pivotal role of 5′-AMP (5′-adenosine monophosphate) in regulation of rate-limiting enzymes: fructose 1,6-diphosphatase (FDP), phosphofructokinase (PFK), glycogen phosphorylase (GP), colipase (CLPS) and pancreatic lipase related protein 2 (PLRP2) for glucose, glycogen and fat metabolism, respectively. Low temperatures differentially affects key enzymes activity. Adenylate pool equilibrium (ATP+5′-AMP< >2ADP) is regulated by adenylate kinase (AK) and the degradation of 5′-AMP is via AMP demainase (AMPD);

FIGS. 37 a and b demonstrate the role of blood glucose in spontaneous arousal by showing the level of blood glucose and activation of procolipase expression during suspended animation and arousal in mice. FIG. 37 a shows the expression of procolipase in liver mRNA from mice sacrificed when CBT was at 37° C. (Start), SA (suspended animation), arousal by rewarming (SA) and at spontaneous arousal (RF). FIG. 37 b shows the level of blood glucose obtained from mice with CBT at 37° C. (start), SA, arousal by rewarming and at spontaneous arousal. (n=5), * P<0.05.

FIGS. 38 a, b, c and d are HPLC graphs showing adenylates and catabolic products in red blood cells. FIG. 38 a is a control animal with CBT at 37° C. FIG. 38 b is an animal at arousal. FIG. 38 c is an animal in SA. FIG. 38 d is an animal in SA given another injection of 5′-AMP and sacrificed 2 hours later. Peak #1 is ATP; Peak #2 is uric acid; Peak #3 is hypoxanthine; Peak #4 is 5′-AMP and Peak #5 is inosine.

FIGS. 39 a, b and c are bar graphs showing the adenylate ratios in blood, liver and muscle during the three behavior states. FIG. 39 a shows the ratios in blood. FIG. 39 b shows the ratios in muscle. FIG. 39 c shows the ratios in liver. ** p<0.01, * p<0.05, n=5.

### DETAILED DESCRIPTION OF THE INVENTION

Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “subject” or “subjects” as used herein refers to animals, including mammals, laboratory animals such as mice, and preferably humans. As used herein, the term “purified” or “to purify” refers to the removal of contaminants from a sample.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (e.g., Sambrook, et al., Molecular Cloning: A Laboratory Manual, Cold Spring Harbor, 1989).

As used herein, a “pharmaceutically acceptable” component is one that is suitable for use with humans and/or animals without undue adverse side effects (such as toxicity, irritation, and allergic response) commensurate with a reasonable benefit/risk ratio to achieve inducing torpor or hibernation in a subject.

As used herein, the term “therapeutically effective amount” is meant an amount of a compound of the present invention effective to yield a desired therapeutic response. For example, to induce torpor or suspended animation in a subject an effective amount of 5-AMP or 5′-AMP analogue may be provided in a form that maximizes a physiologic response with the lowest dosage. The specific “therapeutically effective amount” will, obviously, vary with such factors as the particular condition being treated, the physical condition of the patient, the type of mammal being treated, the duration of the treatment, the nature of concurrent therapy (if any), the gender and/or weight of the animal, the extent to which inducing torpor or hibernation in a subject is needed or for a required amount of time, for which specific formulations may be prepared and employed depending on the structure of the compounds or its derivatives, its solubility, metabolic half-life, etc.

The term “organ” is used herein in its broadest sense and refers to any part of the body exercising a specific function including tissues and cells or parts thereof, for example, cell lines or organelle preparations. Other examples include circulatory organs such as the heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells, myocytes, nerve cells, brain cells or kidney cells.

The term “5′-AMP analogues” are used herein refers to 5′-AMP compounds, analogues, precursors, metabolites and modifications, preferably synthetic and more preferably non-naturally-occurring analogues that induce a state of torpor or suspended animation in a subject by virtue of their ability to stimulate or induce procolipase (Clps) and/or pancreatic lipase related protein 2 (Plrp2) expression or activity. Such analogues include but are not limited to oligonucleotide, oligonucleoside, nucleoside and nucleotide and precursors of 5′-AMP (such as ADP and ATP) that may be converted by chemical or enzymatic methods into a biologically active and useful 5′-AMP analogue. The modifications may include substitutions or other modifications of a heterocyclic base portion of a nucleoside to give a non-naturally-occurring nucleobase, a sugar portion of a nucleoside, the linker groups, the phosphate group or combinations thereof.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

Invention Overview

Generally, hibernation is used by animals to conserve energy during episodes of food stress such as during winter months.¹ The physiological and biochemical signaling processes that regulate hibernation have been, prior to the present invention, an enigma. In mammals, the circadian clock has been implicated in this role since there is an association between daily torpor (e.g., short hibernation-like state) and the body temperature rhythm.²⁻³ Additional evidence implicating the circadian clock is the observation that photoperiod length regulates daily torpor and body weight of mammals.⁴⁻⁵ The ablation of the suprachiasmatic nucleus (SCN), the central circadian clock synchronizer, abolished the torpor rhythm.⁶

Classic hibernation is only observed in rodents such as ground squirrels and large mammals such as bears. However, several strains of laboratory mice can undergo torpor indicating that some basic mechanisms for hibernation are preserved in this organism.⁷⁻⁸

The role of the circadian clock in torpor and the signaling mechanism's that regulate this biological phenomenon in vivo is of great interest. For example, the fat genes encoding for the mouse procolipase (mClps) and pancreatic lipase related protein 2 (Plrp2) are activated in peripheral organs of mice during constant darkness but not during light-dark cycles and is deregulated in mice genetically deficient in circadian clock function. The constant darkness induced enzymes are observed in energy rich tissues and mediate fat (e.g., triacylglycerides) catabolism.

The present invention provides 5′-AMP (e.g., synthetic or natural) and analogues to induce torpor and stimulate the expression of Clps in the peripheral organs, to thereby induce a state of torpor or suspended animation. The present invention therefore provides a mechanism that allows the regulation of metabolic function through the administration of 5′-AMP or its analogues, which serve to deregulate the body's temperature regulating mechanisms, leading to a reduction in core body temperature (CBT). It is postulated by the present inventors that the application of the 5′-AMP shifts the body's metabolism from a primarily glycolytic metabolism to a fatty acid metabolism, as evidenced by an induction in Clps expression. This signal then leads to deregulation of the body's temperature regulating mechanism, which, in turn, leads to a reduction in CBT as the body temperature seeks equilibrium with the ambient environmental temperature (AET). Once the body temperature reaches about 31° C. or lower, a state of torpor or suspended animation is achieved.

Typically when an animal's body temperature is so reduced under non-hibernating conditions, the animal will begin to exhibit dangerous cardiac arrhythmias, ultimately leading to cardiac fibrillation and death. However, in subjects treated with high doses of 5′-AMP, similar to what is observed in hibernating animals, reductions in CBT are not seen to result in serious complications or adverse reactions such as cardiac arrhythmias. It is believed by the inventors that the shift to a fatty acid metabolism away from a glycolytic metabolism plays a role in this protective effect.

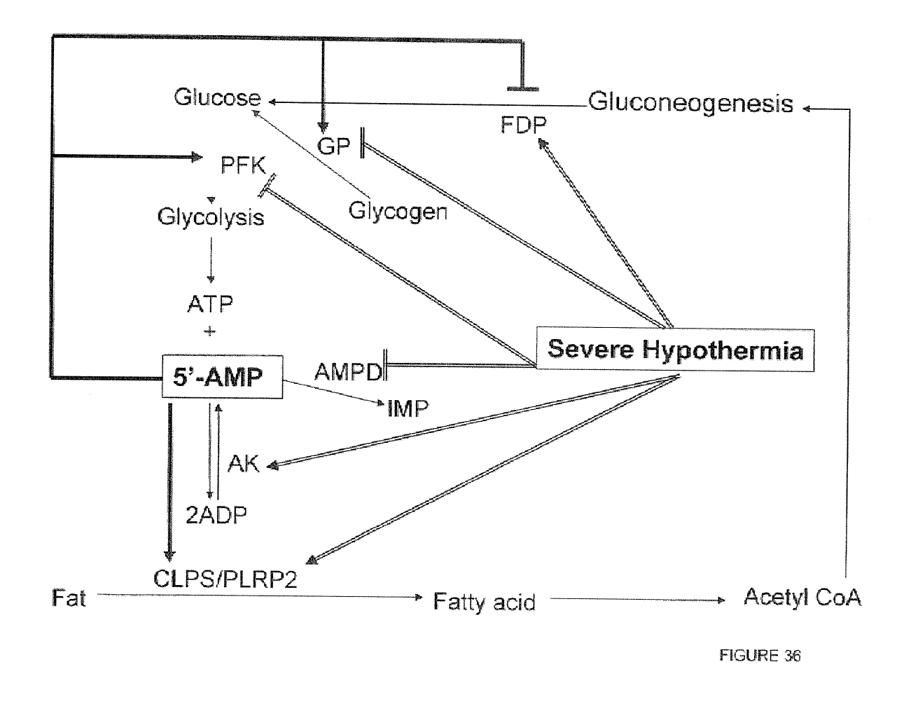

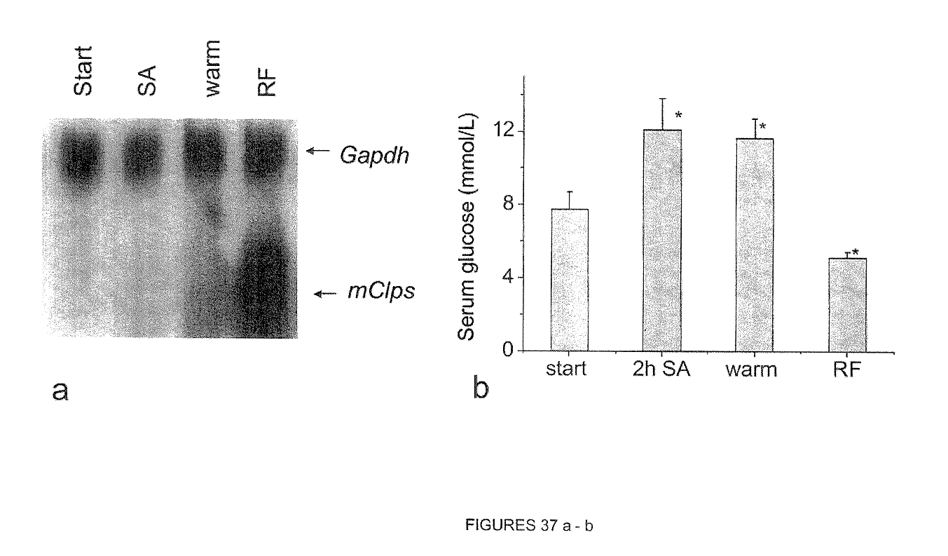

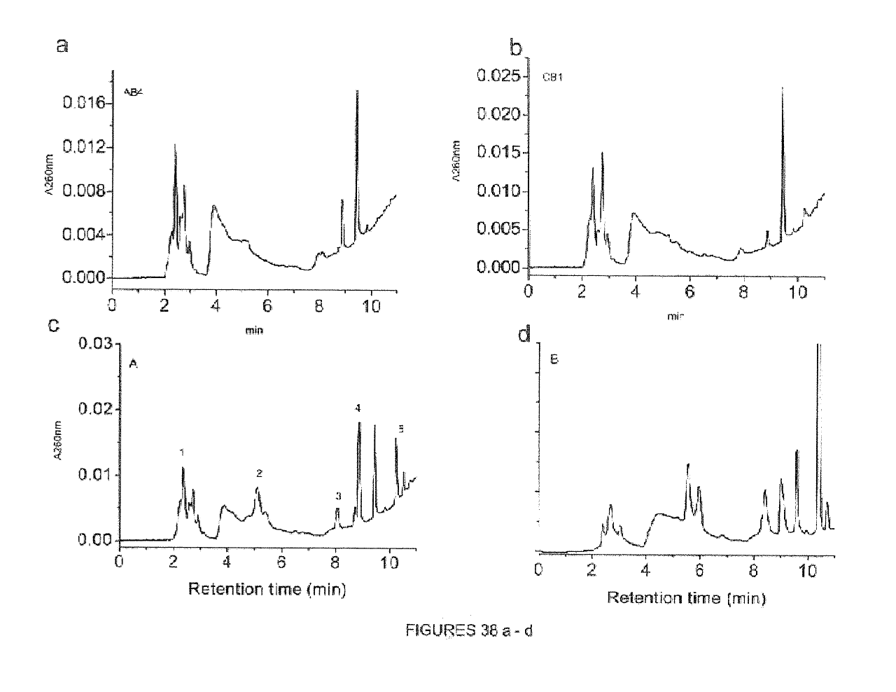

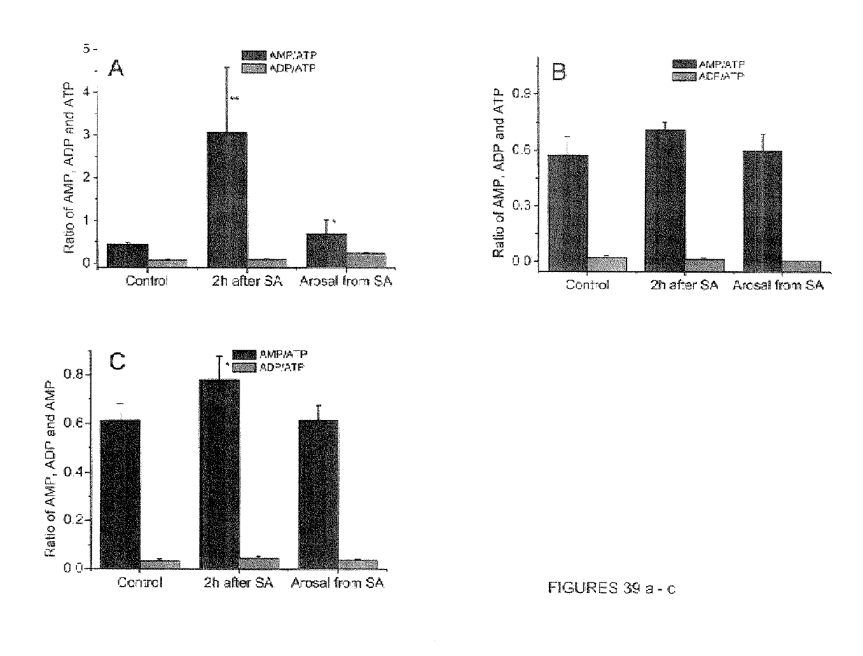

By way of illustration as depicted in FIG. 36, the inventors theorize that the administration of high doses of 5′-AMP to a subject will perturb the adenylate pool equilibrium, which could be corrected by decreasing cellular ATP production. This, in turn, compromises thermoregulatory defenses resulting in a drop in CBT. Severe hypothermia has differential affects on key enzyme activities including those that are allosterically regulated by 5′-AMP that maintain glucose homeostasis. Activity of adenylate kinase, an enzyme that regulates the adenylate pool equilibrium is insensitive to low temperature. In contrast, activity of AMP deaminase, an enzyme that degrades 5′-AMP to inosine monophophate (IMP) is inhibited by low temperature. Therefore, in severe hypothermia, the high 5-AMP levels cannot be degraded and consequently ATP production remained suppressed and CBT remains low thereby SA continues. ATP production in severe hypothermia is primarily through glycolysis. However, the activity of glycogen phosphorylase (GP), the rate-limiting enzyme that degrades stored glycogen into glucose 1-phosphate is inhibited by low temperature. Without stored glycogen as a glucose source, phosphofructose kinase (PFK), the rate-limiting enzyme for glycolysis is repressed by low temperature to conserve blood glucose.

To maintain blood glucose without glycogenolysis, 5′-AMP activates procolipase/pancreatic lipases expression. Low temperature does not inhibit colipase/pancreatic lipases activity, which degrades stored fat into fatty acids. Acetyl-CoA from fatty acids oxidation in turn fuel gluconeogenesis to produce glucose for glycolysis. Activity of fructose-1,6 diphosphatase (FDP), the rate-limiting enzyme of gluconeogenesis is insensitive to low temperature. Even at severe hypothermic state, the low level of glycolysis generates ATP that eventually tip the adenylate pool equilibrium away 5′-AMP. As the ATP level rises, a gradual rise in CBT in SA was observed since there is now energy for thermo-regulatory defenses. Once CBT rose above 17° C., enzymes that were inhibited by severe hypothermia have gradually regained its activities. This further activates thermogenesis mechanism's illustrated by intense shivering, to restore cellular ATP levels and return adenylate pool to its original equilibrium ratio. The inventors' studies showed that the ability to enter SA is not unique to hibernators and can be achieved in non-hibernating animals. The universal role of adenylate pool equilibrium (ATP+5′-AMP< >2ADP) in energy regulation of all living organisms indicates as well that humans could also safely undergo reversible SA when given 5′-AMP and in low AET.

The surprising ability of exogenously added 5′-AMP to permit safe reductions in CBT is an important aspect of the present invention. For example, it is well established that, if CBT can be lowered, the cellular damage from surgical procedure or from a trauma (e.g., an injury as a result of accident, injury or combat) can be reduced significantly. The inventors theorize that this is due to a reduction in the metabolic activity of the cells due to the hypothermic state. The present invention provides physiologically activated or synthetic 5′-AMP to induce torpor which allowed CBT to match closely to ambient room temperature.

Another application of the present invention includes a treatment for heart arrhythmias. The present inventors recognized that 5′-AMP slows the heart rate when given at high concentrations. When compared with one treatment which is known in the art and used for such purposes (e.g., adenosine), adenosine's water solubility is very low unlike 5′-AMP which highly water-soluble. In addition, unlike adenosine, 5′-AMP does not cross the blood brain barrier, and, hence, does not exhibit undesirable neurological effects.

Still another application of the present invention is the treatment for obesity. The present inventors recognized that 5′-AMP induces procolipase expression in all peripheral organs sampled. The procolipase encodes for two peptides, e.g., when cleaved procolipase polypeptide produces colipase and an N-terminal pentapeptide known as enterostatin. Enterostatin is a known satiety inhibitor in animals and human. The present inventors recognized that injections of 5′-AMP induces procolipase expression in all peripheral organs sampled and therefore modulated enterostatin production in vivo. Enterostatin acts naturally on satiety. This is corroborated by studies by the present inventors in which mice kept in constant darkness were observed to have high procolipase expression and reduced food and water intake compared with animals kept in light-dark cycle.

The present invention may be used as a treatment for Type-2 insulin resistant diabetes by regulating human blood glucose level. For example, the 5′-AMP activates procolipase expression is directly linked to blood glucose levels and that 5′-AMP is an allosteric regulator of several key metabolic enzymes (e.g., PFK, FDP and GP) that regulates the body glucose and glycogen levels.

The present invention further provides a mechanism for controlling mammalian behavior and its biological cascades, e.g., constant darkness activates Clps and Plrp2 expression illustrates the potency of this signal. The inventors discovered that activation of these fat catabolism genes in constant darkness mice is mediated by a circadian regulated circulating molecule identified as 5′ adenosine monophosphate (5′-AMP). For example, injection of synthetic 5′-AMP into mice induced mClps expression and at high dosage put the animal into torpor. Circadian-deficient animals displayed an enhanced torpor state in response to 5′-AMP, confirming the endogenous clock role in this molecular cascade. Both food and environmental stress mediate the torpor response of constant darkness mice demonstrating that circulating 5′-AMP functions as an energy regulator. The potency of 5′-AMP in mediating torpor is illustrated by its effects on mice.

In certain embodiments, the present invention further relates to a method and pharmaceutical or veterinary composition for arresting, protecting and/or preserving organs, in particular the heart during open-heart surgery, cardiovascular diagnosis or therapeutic intervention.

The present invention also provides a method for alleviating a disease state in a subject believed to be responsive to treatment with a 5′-AMP or a 5′-AMP analogue by administering to the subject a therapeutic amount a 5′-AMP or analogue. The disease state may be a fever, a heatstroke, eating disorders, anxiety, a seizure, epilepsy, insomnia and sleeping disorders, asthma, diabetes, cardiac arrhythmia, stroke, obesity, hypertension, hyperthyroidism, hypothyroidism and combinations thereof.

In addition, the present invention provides a method for inducing a state of torpor or suspended animation in a subject in need of medical treatment by administering to the subject in need of medical treatment a therapeutic amount a 5′-AMP or analogue to reduce the core body temperature, reduce the external body temperature, reduce the metabolic rate, reduce the heart rate and combinations thereof. This may be used to reduce the core body temperature of a subject and in turn benefit the subject. In addition, the heart rate and metabolism may be altered to aid the subject. In these cases the subject may be any mammal and in particular humans.

The present invention also provides a method of treating insomnia by administering a pharmaceutically effective amount of 5′-AMP or analogue to a subject in a concentration sufficient to induce sleep. For example, a pharmaceutical composition is provided by the present invention. The pharmaceutical composition includes a pharmaceutically effective amount of 5′-AMP or analogue sufficient to produce a hibernation state, a reduction in the core body temperature, an inotropic effect on the heart, a decrease in cell growth, a reduction in metabolic rate, a reduction in the blood glucose levels and combinations thereof.

A method of altering metabolic activity in a subject by administering a pharmaceutically effective amount of 5′-AMP or 5′-AMP analogue to a subject, wherein the 5′-AMP or analogue alters the activity of one or more metabolic enzymes selected from procolipase, pancreatic lipase, pancreatic lipase related protein, phosphofructose kinase, fructose 1,6 diphosphatase, glycogen phosphorylase, and combinations thereof is provided by the present invention.

The present invention still further provides a method for arresting, protecting and/or preserving an organ which includes adding a composition which includes effective amounts of 5′-AMP or analogue to a subject having the organ.

The present invention also provides a tranquilizer composition for use in a projectile for inducing hibernation in a subject. The projectile may be a dart, shot, bullet, probe, stint, arrow, pellet, grenade, mortar, sphere or similar projectile and combinations thereof. Furthermore, the tranquilizer composition may be in the form of a solid, powder, liquid, gel, gas, coated nanoparticle or combinations thereof. The tranquilizer composition may be coated onto, incorporated into or in communication with other compounds and components, e.g., a 5′-AMP coated nanoparticle, or a 5′-AMP vapor interspursed with a smoke grenade or flash grenade. The tranquilizer composition includes a pharmaceutically effective amount of 5′-AMP or analogue and a pharmaceutically acceptable carrier.

The composition of the present invention may be adapted to many different applications including anti-terrorism security systems for use on vehicles (e.g., automobiles, planes, busses, cargo trucks, trains, boats, ships, etc.) and buildings (e.g., offices, banks, federal buildings, courts, headquarters factories and so forth). In addition, the composition of the present invention may be adapted for use in jails and correction facilities to provide a safe and effective mechanism to control individuals, e.g., riots. As an example, the present invention may be incorporated into an aircraft as an anti-terrorism security system.

An aircraft anti-terrorism security system for inducing torpor in one or more subjects on the aircraft is provided. The anti-terrorism security system includes an aircraft having a fuselage, a cockpit in the fuselage, and a cabin in the fuselage adjacent to the cockpit. A pressurized security system is disposed within the aircraft and includes one or more outlets in the fuselage. The pressurized security system includes a pharmaceutically effective amount of 5′-AMP or analogue and a propellant. One or more activation mechanisms are also provided. The activation of the one or more activation mechanisms results in the release of the 5′-AMP or analogue of 5′-AMP induces into the cockpit, the cabin or both and induces torpor when inhaled by the one or more subjects. In addition, an isolated air system in the cockpit is provided for one or more pilots.

One or more activation mechanisms are also provided and may be hard wired into various points throughout the airplane. Although the activation mechanisms may be hardwired this is not a necessity and wireless devices (e.g., remote, key chain, etc.) may be used and carried by air marshals, pilots, flight crew and so forth. In some embodiments, the one or more outlets are configured to interface with the aircraft air circulation system so that the pharmaceutically effective amount of 5′-AMP or analogue and a propellant is directed at the subjects. One interface method includes the incorporation of outlets directly into the aircraft air circulation system. As there are instances where the subjects may not be located adjacent to the outlet of the aircraft air circulation system, separate outlets may be positioned throughout the aircraft. The isolated air system in the cockpit is provided for one or more pilots may be in the form of an airtight cockpit without outlets for the pressurized security system or a mask attached to a separate air system.

Another potential usage of 5′-AMP is in the early treatment of strokes. Since 5′-AMP can reduce CBT, heart rate, and metabolic rate, the level of bleeding into the brain as a result of a stroke can be reduced after injection of 5′-AMP. In addition reduction of CBT will limit the destruction of cells due to decrease demand of oxygen and other metabolic requirement before, during and after surgery.

Pharmaceutical Compositions

The present invention provides a method of inducing torpor or suspended animation in a subject by administering a pharmaceutically effective amount of 5-AMP or 5′-AMP analogue to a subject. The 5′-AMP or analogue is adapted for administration by subcutaneous injection, intramuscular injection, intravenous injection, intraperitoneal injection, nasal administration, intravaginal administration, intranasal administration, intrabronchial administration, intraocular administration, intraaural administration, intracranial administration, oral consumption, parenteral administration, rectal administration, sublingual administration, topical administration, transdermal administration or combination thereof. The 5′-AMP or analogue is packed into a capsule, caplet, softgel, gelcap, suppository, film, granule, gum, insert, pastille, pellet, troche, lozenge, disk, poultice, wafer, creams, lotions, ointments, aerosol sprays, roll-on liquids, roll-on sticks, transdermal patches, subcutaneous implants, pads and combinations thereof. The 5′-AMP or analogue may be disposed for extended release in a biodegradable carrier.

The pharmaceutically effective amount of 5′-AMP or analogue may also include one or more pharmaceutically acceptable carriers. For example, pharmaceutically acceptable carriers include water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

In some embodiments, the pharmaceutically acceptable carrier includes an aerosol propellant selected from propane, butane, isobutene, pentane, isopropane, fluorocarbons, dimethylether and mixtures thereof. However, other aerosol propellants known to the skilled artisan may be used, e.g., ethers, dimethylether C₁-C₆ saturated hydrocarbons, propane, butane, isobutene, pentane, isopropane, hydrofluorocarbons, fluorocarbons and mixtures thereof.

Depending on the particular type of administration being used for subject a variety of different delivery methods and constructs will be used. Common delivery methods of 5′-AMP or analogue include a chewable tablet, a solid, a dissolvable or disintegrating tablet, a liquid, a gel, a tab, a capsule, a powder, a lotion, a cream, a gum, a lozenge and combinations thereof. Furthermore, the composition may include least one agent selected from the group consisting of emollients, water, inorganic powders, foaming agents, emulsifiers, fatty alcohols, fatty acids and combinations thereof.

The pharmaceutical composition is adapted for administration by subcutaneous injection, intramuscular injection, intravenous injection, intraperitoneal injection, nasal administration, intravaginal administration, intranasal administration, intrabronchial administration, intraocular administration, intraaural administration, intracranial administration, oral consumption, parenteral administration, rectal administration, sublingual administration, topical administration, transdermal administration or combination thereof. The pharmaceutical composition may be packed into a capsule, caplet, softgel, gelcap, suppository, film, granule, gum, insert, pastille, pellet, troche, lozenge, disk, poultice, wafer, creams, lotions, ointments, aerosol sprays, roll-on liquids, roll-on sticks, transdermal patches, subcutaneous implants, pads and combinations thereof. Furthermore, the pharmaceutical composition may be disposed for extended release in a biodegradable carrier.

The pharmaceutical composition containing a pharmaceutically effective amount of 5′-AMP or 5′-AMP analogue may also include one or more pharmaceutically acceptable carriers. For example, pharmaceutically acceptable carriers include water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

In some embodiments, the pharmaceutically acceptable carrier includes an aerosol propellant selected from propane, butane, isobutene, pentane, isopropane, fluorocarbons, dimethylether and mixtures thereof. However, other aerosol propellants known to the skilled artisan may be used, e.g., ethers, dimethylether C₁-C₆ saturated hydrocarbons, propane, butane, isobutene, pentane, isopropane, hydrofluorocarbons, fluorocarbons and mixtures thereof.

Depending on the particular type of administration, a variety of different delivery methods and constructs will be used. Common delivery methods of 5′-AMP or analogue include a chewable tablet, a solid, a dissolvable or disintegrating tablet, a liquid, a gel, a tab, a capsule, a powder, a lotion, a cream, a gum, a lozenge and combinations thereof. Furthermore, the composition may include least one agent selected from the group consisting of emollients, water, inorganic powders, foaming agents, emulsifiers, fatty alcohols, fatty acids and combinations thereof.

The pharmaceutically acceptable carrier may be water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

The present invention provides an aerosol composition adapted for inducing torpor in a subject. The aerosol composition includes a pharmaceutically effective amount of 5′-AMP or analogue and a propellant. In some embodiments, the aerosol composition is in the form of an inhaler; however, other systems may be used.

The pharmaceutical composition containing a pharmaceutically effective amount of 5′-AMP or an analogue of 5′-AMP may also include one or more pharmaceutically acceptable carriers or other active agents. For example, pharmaceutically acceptable carriers include water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof. Examples of other active agents include are listed herein and known to the skilled artisan.

In some embodiments, the pharmaceutically acceptable carrier includes an aerosol propellant selected from propane, butane, isobutene, pentane, isopropane, fluorocarbons, dimethylether and mixtures thereof. However, other aerosol propellants known to the skilled artisan may be used, e.g., ethers, dimethylether C₁-C₆ saturated hydrocarbons, propane, butane, isobutene, pentane, isopropane, hydrofluorocarbons, fluorocarbons and mixtures thereof.

The present invention also provides an aerosol for reducing the core body temperature of a subject. An aerosol is particularly advantageous with regards to children and animals, which are sometimes uncooperative with the administration of medicines.

The present invention also provides an aerosol for reducing the core body temperature of a subject. The aerosol includes an aerosol container having one or more sides and an activation mechanism. The aerosol container may take any convenient form. In some instances the aerosol container will take the form of an aerosol sprayer similar to a hairspray bottle, while in other embodiments the aerosol container may in the form of a sphere. Within the aerosol container is a pharmaceutically effective amount of 5′-AMP or analogue disposed.

The pharmaceutical composition containing a pharmaceutically effective amount of 5′-AMP or 5′-AMP analogue may also include one or more pharmaceutically acceptable carriers or other active agents. In addition, the aerosol container includes a propellant disposed therein. In some embodiments, the pharmaceutically acceptable carrier includes an aerosol propellant selected from propane, butane, isobutene, pentane, isopropane, fluorocarbons, dimethylether and mixtures thereof. However, other aerosol propellants known to the skilled artisan may be used, e.g., ethers, dimethylether C₁-C₆ saturated hydrocarbons, propane, butane, isobutene, pentane, isopropane, hydrofluorocarbons, fluorocarbons and mixtures thereof.

Pharmaceutical compositions of the present invention suitable for injectable use include sterile aqueous solutions or dispersions and sterile powders for the extemporaneous preparation of sterile injectable solutions or dispersion. In all cases, the composition must be sterile and must be fluid to the extent that easy syringability exists. The carrier may be a solvent or dispersion medium containing, e.g., water, ethanol, polyol (e.g., glycerol, propylene glycol, and liquid polyethylene glycol, and the like), suitable mixtures thereof, and vegetable oils.

Sterile injectable solutions of the present invention may be prepared by incorporating the present invention in the required amount in an appropriate solvent with one or a combination of ingredients enumerated herein or known to the skilled artisan. Generally, dispersions are prepared by incorporating the therapeutic compound into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the therapeutic compound) plus any additional desired ingredient from a previously sterile-filtered solution thereof.

Pharmaceutical compositions of the present invention are also suitable for oral administration, e.g., with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied as will be known to the skilled artisan. The amount of the therapeutic compound in such therapeutically useful composition is such that a suitable dosage will be obtained.

Parenteral compositions of the present invention may be in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier.

Solutions of the present invention may be prepared in water suitably mixed with a surfactant, such as hydroxypropylcellulose. Dispersions may also be prepared in glycerol, liquid polyethylene glycols, and/or mixtures thereof and/or in oils. Under ordinary conditions of storage and/or use, these preparations contain a preservative to prevent the growth of microorganisms.

For oral, buccal, and sublingual administration, the pharmaceutical composition of the present invention may be administered as either solutions or suspensions in the form of gelcaps, caplets, tablets, capsules or powders. For rectal administration, the compounds of the invention may be administered in the form of suppositories, ointments, enemas, tablets and creams for release of compound in the intestines, sigmoid flexure and/or rectum. For example, when making a suppository a beeswax/glycerol composition may be used to form a body meltable suppository for transrectal or transurethral delivery.

Other additives conventionally used in pharmaceutical compositions may be included, which are well known in the art. Such additives include, e.g.: anti-adherents (anti-sticking agents, glidants, flow promoters, lubricants) such as talc, magnesium stearate, fumed silica), micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate.

Other additives include, binders (e.g., adhesives), i.e., agents that impart cohesive properties to powdered materials through particle-particle bonding, such as matrix binders (e.g., dry starch, dry sugars), film binders (e.g., PVP, starch paste, celluloses, bentonite and sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, e.g., PVP, PEG, vinyl pyrrolidone copolymers, pregelatinized starch, sorbitol, and glucose).

For certain actives it may be useful to provide buffering agents (or bufferants), where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid.

The liquid dosage form may also contain one or more chelating agents. Examples of chelating agents include, e.g., polyacrylic acid, citric acid, edetic acid, disodium edetic acid, and the like. The chelating agent may be co-delivered with the active agent in the environment of use to preserve and protect the active agent in situ. Such chelating agents may be combined with the liquid, active agent formulation in the porous particles, or the chelating agents may be incorporated into the drug layer in which the porous particles are dispersed.

The liquid formulation may also include one or more surfactants, e.g., nonionic, anionic and cationic surfactants, or combinations thereof. Examples of nontoxic, nonionic surfactants suitable for forming a liquid-based formulation include, e.g., alkylated aryl polyether alcohols known as Triton™; polysorbates such as polysorbate 80; polyethylene glycol tertdodecyl throether available as Nonic™; fatty and amide condensate or Alrosol™; aromatic polyglycol ether condensate or Neutronyx™; fatty acid alkanolamine or Ninol™; sorbitan monolaurate or Span™; polyoxyethylene sorbitan esters or Tweens™; sorbitan monolaurate polyoxyethylene or Tween 20™; sorbitan mono-oleate polyoxyethylene or Tween 80™; polyoxypropylene-polyoxyethylene or Pluronic™; polyglycolyzed glycerides such as Labraosol, polyoxyethylated castor oil such as Cremophor and polyoxypropylene-polyoxyethylene-8500 or Pluronic™. By way of example, anionic surfactants include, e.g., sulfonic acids and the salts of sulfonated esters such as sodium lauryl sulfate, sodium sulfoethyl oleate, dioctyl sodium sulfosuccinate, cetyl sulfate sodium, mylistyl sulfate sodium; sulfated esters; sulfated amides; sulfated alcohols; sulfated ethers; sulfated carboxylic acids; sulfonated aromatic hydrocarbons; sulfonated ethers; and the like. Cationic surface active agents for use with liquid formulations, include, e.g., cetyl pyridinium chloride; cetyl trimethyl ammonium bromide; diethylmethyl cetyl ammonium chloride; benzalkonium chloride; benzethonium chloride; primary alkylammonium salts; secondary alkylammonium salts; tertiary alkylammonium salts; quaternary alkylammonium salts; acylated polyamines; salts of heterocyclic amines; palmitoyl carnitine chloride, behentriammonium methosulfate, and the like. Surfactants with be provided generally, from 0.01 part to 1000 parts by weight of surfactant, per 100 parts of the active agent; however, the skilled artisan will recognize that other concentrations and other parts by weight of surfactant and parts per 100 parts of the active agent may be used.

Examples of active agent for use with the present invention include: Analgesic anti-inflammatory agents e.g., acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like. Agents having an action on the central nervous system, e.g., sedatives, hypnotics, antianxiety agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocaine, benzocaine, fentanyl, nicotine, and the like. Local anesthetics such as, benzocaine, procaine, dibucaine, lidocaine, and the like.

Antihistaminics or antiallergic agents e.g., diphenhydramine, dimenhydrinate, perphenazine, triprolidine, pyrilamine, chlorcyclizine, promethazine, carbinoxamine, tripelermamine, brompheniramine, hydroxyzine, cyclizine, meclizille, clorprenaline, terfenadine, chlorpheniramine, and the like. Anti-allergenics such as, antazoline, methapyrilene, chlorpheniramine, pyrilamine, pheniramine, and the like. Decongestants e.g., phenylephrine, ephedrine, naphazoline, tetrahydrozoline, and the like. Antipyretics e.g., aspirin, salicylamide, non-steroidal anti-inflammatory agents, and the like. Antimigrane agents e.g., dihydroergotamine, pizotyline, and the like. Acetonide anti-inflammatory agents, e.g., hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprofen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like.

Muscle relaxants such as, tolperisone, baclofen, dantrolene sodium, cyclobenzaprine. Steroids such as, androgenic steroids, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlonnadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β₂-adrenergic agonists, such as, albuterol, terbutaline, metaproterenol, ritodrine, carbuterol, fenoterol, quinterenol, rimiterol, solmefamol, soterenol, tetroquinol, and the like. Sympathomimetics such as, dopamine, norepinephrine, phenylpropanolamine, phenylephrine, pseudoephedrine, amphetamine, propylhexedine, arecoline, and the like. Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlommethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE1, PGE2α, and PGF2α, and the PGE1 analog misoprostol. Antispasmodics such as, atropine, methantheline, papaverine, cinnamedrine, methscopolamine, and the like. Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenyloin, and the like.

Agents for dizziness such as, isoprenaline, betahistine, scopolamine, and the like. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like. Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranquilizers such as, chlorprathixene, thiothixene, haloperidol, bromperidol, loxapine, and molindone, as well as, those agents used at lower doses in the treatment of nausea, vomiting, and the like. Antitumor agents such as, 5-fluorouracil and derivatives thereof, krestin, picibanil, ancitabine, cytarabine, and the like. Anti-estrogen or anti-hormone agents such as, tamoxifen or human chorionic gonadotropin, and the like. Miotics such as pilocarpine, and the like. Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like. Mydriatics such as, atropine, cyclopentolate, homatropine, scopolamine, tropicamide, eucatropine, hydroxyamphetamine, and the like. Psychic energizers such as 3-(2-aminopropyl)indole, 3-(2-aminobutyl)indole, and the like.

Antidepressant drugs such as, isocarboxazid, phenelzine, tranylcypromine, imipramine, amitriptyline, trimipramine, doxepin, desipramine, noltriptyline, protriptyline, amoxapine, maprotiline, trazodone, and the like. Anti-diabetics such as, insulin, and anticancer drugs such as, tamoxifen, methotrexate, and the like. Anorectic drugs such as, dextroamphetamine, methamphetamine, phenylpropanolamine, fenfluramine, diethylpropion, mazindol, phentermine, and the like. Anti-malarials such as, the 4-aminoquinolines, alphaminoquinolines, chloroquine, pyrimethamine, and the like. Anti-ulcerative agents such as, misoprostol, omeprazole, enprostil, and the like. Antiulcer agents such as, allantoin, aldioxa, alcloxa, N-methylscopolamine methylsulfate, and the like. Antidiabetics such as insulin, and the like.

The drugs mentioned above may be used in combination as required. Moreover, the above drugs may be used either in the free form or, if capable of forming salts, in the form of a salt with a suitable acid or base. If the drugs have a carboxyl group, their esters may be employed.

### EXAMPLES

The following examples are included to illustrate studies involved in the development of the invention and to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

### mClps and mPlrp2 Expression are Activated by Constant Darkness

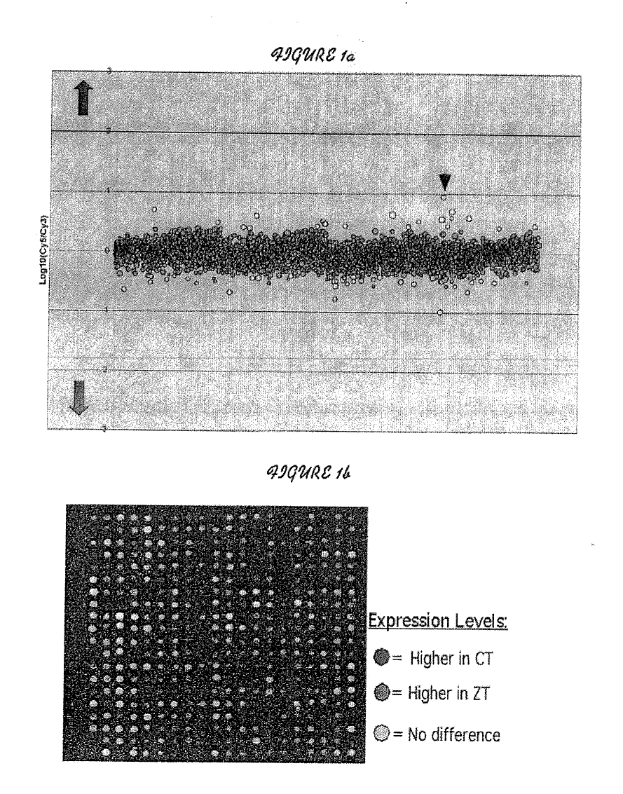

Generally, during hibernation, an animal departs from light-dark and enters a constant darkness environment.⁹ FIG. 1a is the analysis of the cDNA micro-array image in FIG. 1b to determine whether there is a differential pattern of gene expression in livers of mice kept in constant darkness versus light-dark environment. The genes analyzed include the gene that encodes for CLPS the enzymatic partner of PLRP2 required in the gastrointestinal organs for dietary fat degradation.¹⁰ The observed mClps expression in the liver was unexpected since previous studies have demonstrated that expression of this gene is tissue specific and restricted to pancreas and the gastrointestinal organs.¹⁰⁻¹¹

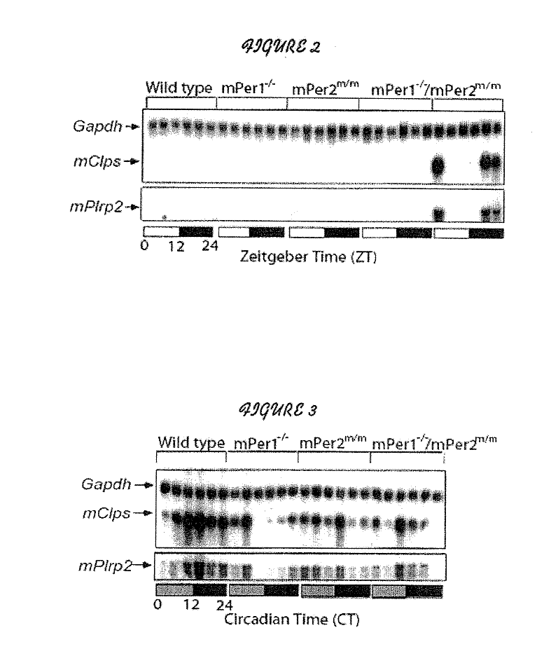

FIG. 2 is an image of a Northern blot that illustrates mClps expression in liver mRNA of wild type, mPer1 null (mPer1^{-/-}), mPer2 mutant (mPer2^{m/m}), and double mutant for mPER1 and mPER2 deficiency (mPer1^{-/-}/mPer2^{m/m}) mice during zeitgeber time (ZT).¹²⁻¹³ Northern blot analysis showed no mClps expression in liver mRNA of light-dark mice with the following genotype: wild type, mPer1^{-/-}, and mPer2^{m/m}. However, in three light-dark mice of the mPer1^{-/-}/mPer2^{m/m} genotype that are completely deficient in circadian clock function, robust mClps expression was observed. Therefore, the expression of mClps is likely under circadian control which is confirmed by the constant darkness studies. Constant darkness activates mClps and mPlrp2 expression in mouse livers. Expression of mClps and mPlrp2 in light-dark mice by Northern blot analysis. Note: for mPer1^{-/-}/mPer2^{m/m} samples, the first 6 lanes from left to right are the corresponding mRNA from kidney tissues.

FIG. 3 is an image of a Northern blot that illustrates mClps expression in liver from constant darkness mice. Northern blot analysis revealed that in the four genotypes, mClps was expressed in the circadian times (CT) studied. Expression of mClps and mPlrp2 in constant darkness mice: Liver RNAs were obtained from wild type, mPer1^{-/-/mPer}2^{m/m} and mPer1^{-/-/mPer}2^{m/m} mice about every 4 hours in either light-dark or constant darkness as described herein and Gapdh mRNA was monitored as an internal control. Furthermore, the mClps expression in wild type constant darkness mice displayed a robust circadian pattern whereas in the circadian deficient mPer1^{-/-}, mPer2^{m/m} and mPer1^{-/-}/mPer2^{m/m} animals, this oscillating profile was deregulated. Multiple days molecular analysis further confirmed that the circadian clock regulate expression of mClps in constant darkness mice, e.g., see the Northern blot image of FIG. 4a that illustrates mClps expression in liver. The expression of mClps was coordinated with expression of its enzymatic partner mPlrp2. Northern blot analysis revealed that mPlrp2 expression patterns were identical to those of mClps as seen in FIGS. 2 and 3. Thus, molecular and genetic studies demonstrated that expression of both mClps and mPlrp2 in liver are under circadian control and are activated by constant darkness. mClps mediates lipid catabolism and is induced by constant darkness via a blood signal.

FIG. 4b is an image of a Northern blot that illustrates the circadian phase of colipase expression in the various peripheral organs of constant darkness mice was similar. Peripheral organs of constant darkness and light-dark mice for mClps expression were analyzed using a Northern blot to showed that mClps expression was only found in pancreas and stomach in light-dark mice and thereby consistent with its primary role in dietary fat degradation.¹⁰⁻¹¹ By contrast in constant darkness mice, robust expression of mClps was observed in skeletal muscle, adipose tissue, heart, liver, and lung in addition to the dietary organs. No expression was observed in brain and kidney as seen in the image of the Northern blot in FIG. 6. Northern blot analysis of mClps expression in peripheral tissues sampled at ZT12.

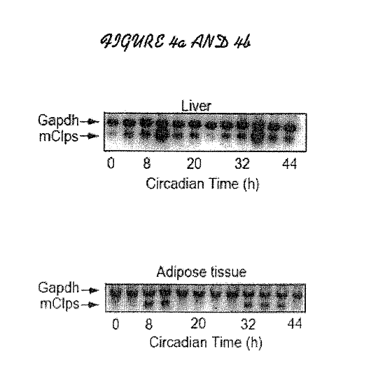

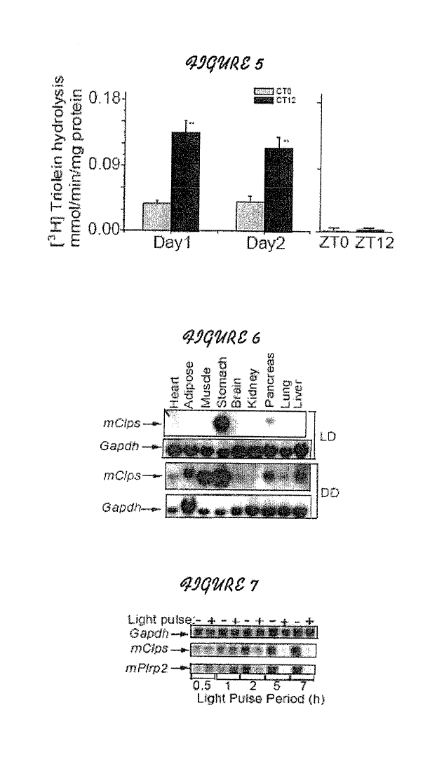

FIG. 5 is a graph illustrating the hydrolysis triacylglycerol substrate analog by liver protein per time that illustrates mClps expression in liver is involved in fat degradation, colipase activity in extracts measured via the release of radiolabel free fatty acid from a triacylglycerol substrate, e.g., [³H] Triolein. Liver extracts obtained from light-dark mice displayed no colipase activity towards triolein (data not shown). By contrast, liver extracts of mice sacrificed on consecutive constant darkness cycles displayed a circadian pattern of colipase activity. Gapdh mRNA was monitored as an internal control. Colipase activity in light-dark and constant darkness mouse livers. Error bars indicate the SEM (n=3).

The kinetics of light inhibition of colipase expression in constant darkness mice has also been examined to determine the signaling mechanism. Northern blot analysis revealed that both mClps and mPlrp2 expression remained high during the first hour of light exposure but decline to basal level after about 5 to 7 hours of light exposure as seen in FIG. 7. Light inhibition of mClps and mPlrp2 expression in DD mice starting at CT7 were determined by Northern blot analysis. A doublet band seen with mPlrp2 probe indicate cross reactivity with its isoform, mPlrp1. Gapdh mRNA was monitored as an internal control. By contrast, constant darkness mice that were not exposed to light displayed a robust level of mClps and mPlrp2 expression. Taken together with the broad expression pattern in constant darkness mice, colipase expression in constant darkness mice is likely mediated by a circulatory signal. The putative circulatory signal(s) may act either as a repressor or as an activator of mClps and mPlrp2 expression during the light-dark or constant darkness cycle, respectively. For example, a putative activator when injected into light-dark mice induces expression of the mClps gene. Similarly, a putative repressor when injected into constant darkness mice inhibits mClps expression.

### Example 2

### A Circadian Regulated Molecule is Elevated in the Blood of Constant Darkness Mice

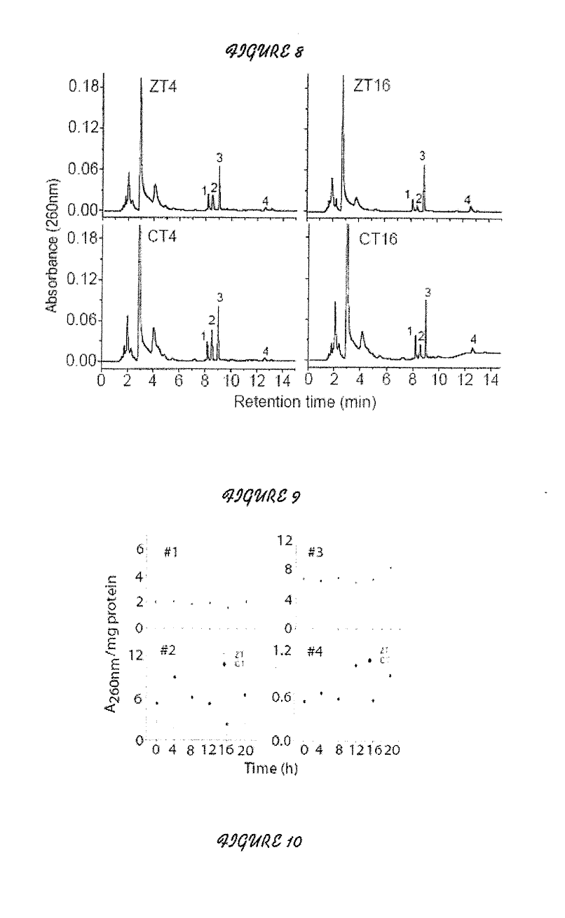

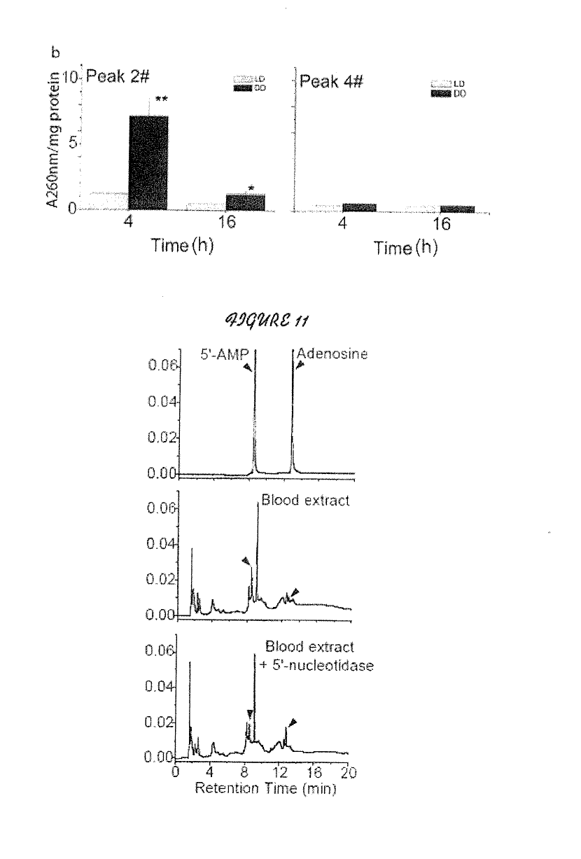

The non-polypeptide aqueous phase organic fraction of blood extracts obtained from mice at various ZT and CT that were analyzed to identify the putative circulatory mediator. FIG. 8 is a chromatogram illustrating the retention times various peaks analyzed by reverse phase HPLC. Representative profiles of high pressure liquid chromatography (HPLC) analysis of blood extracts taken from light-dark mice at ZT0 and ZT12. Note the diurnal and circadian profile of peak #2.

After resolution of extracts by reverse phase HPLC, excluding the unresolved peaks in the initial void volume with retention time below about 5 minutes, there were four highly reproducible peaks (e.g., labeled #1, #2, #3 and #4). One peak (#2) had a robust diurnal and circadian pattern in both ZT and CT samplings as seen in the FIG. 8, while peak #4 may have a weak apparent diurnal and circadian variation. Analysis of peaks #1 and #3 indicated no apparent diurnal pattern as seen in the graph of FIG. 9. FIG. 10 is a bar graph of the absorbance at specific times for constant darkness mice and light-dark mice. Quantification of HPLC peak area of #1, #2, #3 and #4 in light-dark and constant darkness mice with respect to their internal protein concentrations (e.g., A₂₆₀ₙₘ/mg protein) with error bars indicate the SD (n=2). The levels of peaks #2 and #4 obtained from constant darkness mice (n=4) were compared to those from light-dark mice (n=4) to illustrate that only peak #2 was substantially higher in constant darkness mice compared to light-dark animals a characteristic consistent with the hypothesized circulatory signal, e.g., see FIGS. 8, 9 and 10.

### Example 3

### The Circadian Regulated Signal is 5′-Adenosine Monophosphate

FIG. 11 is a HPLC chromatogram comparing the retention time of various samples and chemical standard compounds. Spectral scanning of peak #2 revealed a maximum absorbance at 260 nm, suggesting a nucleotide based molecule. Using HPLC analysis of chemically defined nucleotide standards, peak #2's and #4 had retention time of about 8.5 minutes and about 12.5 minutes respectively. These peaks correlated to the retention times of 5′-adenosine monophosphate (5′-AMP) and adenosine, respectively, see FIG. 11 upper panel. Similarly, peak #1 was matched to adenosine 5′-diphosphate (ADP) at about 8 minutes (data not shown). To confirm the identification of peak #2, the samples were treated with snake venom 5′-nucleotidase that has been affinity purified by AMP-agarose chromatography. HPLC analysis revealed that peak #2 was degraded by the 5′-nucleotidase and peak #4 was enhanced. This demonstrated that peak #2 was 5′-AMP and that peak #4 was adenosine, see FIG. 11 middle and lower panels. The retention time for cyclic-AMP, or ATP was approximately about 13 minutes and about 3 minutes, respectively (data not shown) and therefore excluded the possibility that either cyclic-AMP, or ATP were associated with peak #2.

### Example 4

### 5′-AMP Induces mClps Expression and Torpor in Light-Dark Mice

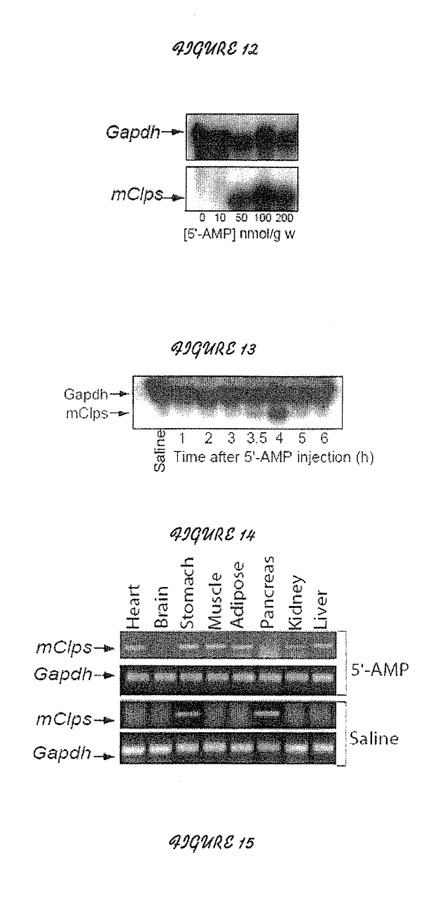

Exogenous 5′-AMP was injected into light-dark mice to test induction of mClps expression and to demonstrate that 5′-AMP is the regulatory signal. FIG. 12 is an image of a Northern blot analysis that demonstrates that 5′-AMP at various concentrations induced mClps expression in the liver. Northern blot of liver RNA for mClps expression in wild type mice injected with saline or various dosages of 5′-AMP at ZT8. Gapdh level was monitored as internal control.

FIG. 13 is an image of a Northern blot that illustrates the induction of mClps expression was observed between about 3.5 and about 4 hours after 5′-AMP was injected. FIG. 14 is an image of a gel that illustrates 5′-AMP induction of mClps expression could be detected in all peripheral tissues sampled except the brain using RT-PCR techniques. Ecto-5′nucleotidase is glycosyl-phosphatidylinositol anchored on the plasma membrane converts 5′-AMP to adenosine extracellularly.¹⁴

FIG. 15 is a Northern blot examining the intracellular action of 5′-AMP via adenosine receptors or transporters. FIG. 15 illustrates that adenosine injected into light-dark mice also induced mClps expression in the liver, but the effect was concentration-gated. In contrast, NECA, a potent adenosine receptor agonist did not induce colipase expression in liver when injected into light-dark mice (data not shown).

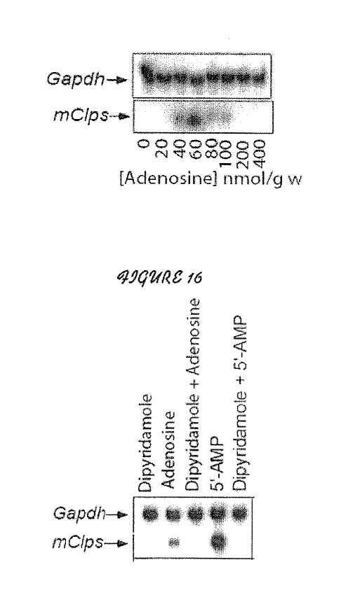

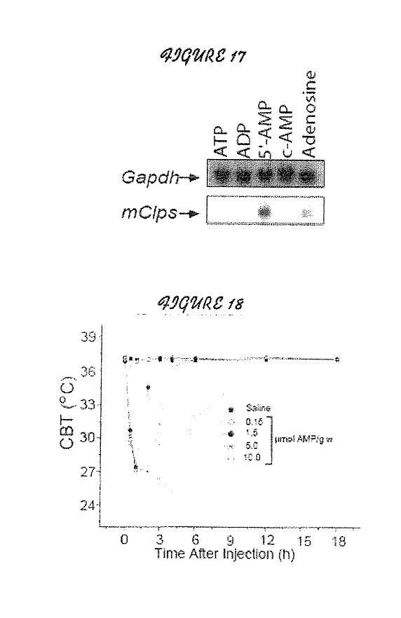

FIG. 16 is a Northern blot examining the blocking of adenosine induction of colipase by dipyridamole, a potent inhibitor of nucleoside transporters.¹⁵ FIG. 17 is a Northern blot analyzing whether other adenine nucleotides could also induce mClps expression in the liver. Mice were injected with similar concentrations of ATP, ADP or c-AMP and Northern blot analysis showed that these nucleotides did not induce mClps expression in the liver. Light-dark mice given a high dosage of 5′-AMP exhibits a body temperature significantly lower than the saline treated mice suggesting that the animals were in torpor. A characteristic feature of torpor is a loss of endothermic control of core body temperature (CBT) at about 37° C. For example, the laboratory mouse is regarded to be in torpor when its CBT is about 31° C. or below.⁷⁻⁸ Therefore, CBT below about 31° C. was used as a quantitative parameter to investigate torpor state. A high dose of 5′-AMP induced apparent sleep activity in mice and their CBT dropped from about 37° C. to about 27° C. during about the first hour (ambient room temperature was 24° C.).

FIG. 18 is a graph of core body temperature verses time after administering 5′-AMP. The length of torpor was dependent on the dosage of 5′-AMP injected. Mice injected with saline showed no temperature fluctuation from 37° C. during the same period. No apparent adverse effects on the torpid mice were observed after their CBT had returned to 37° C. Core body temperature of wild type after injection with saline or various dosages of 5′-AMP. Error bars indicate SEM (n=3).

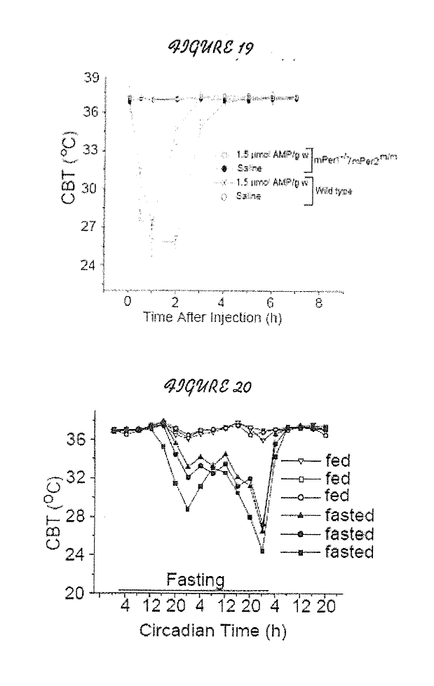

FIG. 19 is a graph of temperature verses time after administering 5′-AMP to examine the circadian clock's role in this mechanism. mPer1^{-/-}/mPer2^{m/m} mice were injected with the same dose of 5′-AMP that induced torpor in wild type mice. CBT measurements in FIG. 19 showed that 5′-AMP induced torpor was more than 2 fold longer in mPer1^{-/-}/mPer2^{m/m} mice than in wild type mice. Core body temperature of wild type and mPer1^{-/-}/mPer2^{m/m} mice after injection with saline or about 1.5 μmol of 5′-AMP per gram body weight. Error bars indicate SEM (n=3). Together, these studies demonstrate that 5′-AMP is the circadian signal that mediates mClps expression in peripheral organs and induces torpor in mice.

### Example 5

### 5′-AMP Regulates Energy Homeostasis of Mice

FIG. 20 is a graph of temperature verses time after administering 5′-AMP to examine the effect of metabolic stress. The behavior of constant darkness mice fed ad libitum were compared to mice that were subjected to metabolic stress conditions. Metabolic stress was generated by short-term food deprivation starting at CT2. During the first twelve hours, CBT sampled about every 4 hours showed minimal variation from about 37° C. in both fed and fasted constant darkness mice at an ambient room temperature of about 23° C. By the second day of fasting, CBT measurement showed that 1 fasted mouse has spontaneously undergone torpor, see FIG. 20. Individual CBT measurements of fed and fasted mice during constant darkness cycle at ambient room temperature (e.g., between about 23° C. and about 24° C.) and at about 4° C. Note the time scale between CT0-CT2 is expanded. However, by the third day all of the fasted mice displayed spontaneous torpor with CBT below about 31° C. while that of fed mice remained at about 37° C.

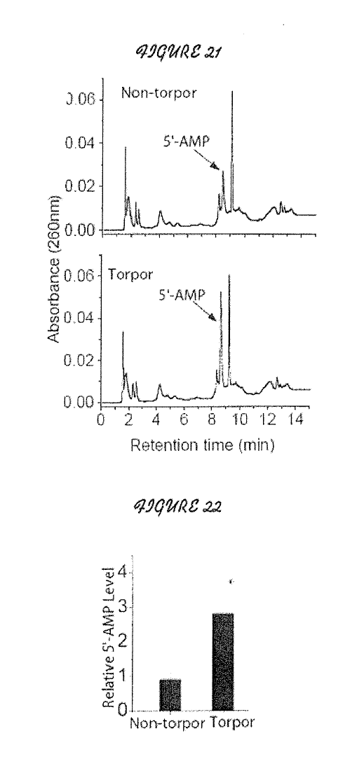

FIG. 21 is a HPLC chromatogram that compares the retention times of 5′-AMP levels in the blood of torpid and non-torpid constant darkness mice. Representative HPLC analysis of blood extract from non-torpid (upper panel) and a torpid mouse (lower panel). HPLC analysis revealed that 5′-AMP levels in torpid mice were highly elevated compared to non-torpid constant darkness animals. Quantification of the relative HPLC peak sizes revealed that 5′-AMP levels were elevated by about 3-fold in torpid constant darkness mice are expressed in the bar graph in FIG. 22. Relative level of 5′-AMP in torpid and non-torpid constant darkness mice. The average value of 5-AMP levels from non-torpid mice is arbitrary set as 1. Error bars indicate SEM (n=3).

Similar studies carried at ambient temperature of about 4° C. showed that the constant darkness mice undergo torpor after one day of fasting and their blood 5′-AMP was also elevated compared with non-torpid controls, e.g., see FIGS. 23a, 23b and 23c are plots demonstrating that under metabolic stress physiological control of 5′-AMP levels induce torpor in constant darkness mice. FIG. 23a is a graph of temperature verses time after administering 5′-AMP to examine the effect of metabolic stress. FIG. 23b is a HPLC chromatogram that compares the retention times of 5′-AMP levels in the blood of torpid and non-torpid constant darkness mice. Quantification of the relative HPLC peak sizes revealed that 5′-AMP levels were elevated by about 3-fold in torpid constant darkness mice are expressed in the graph in FIG. 23c.

### Example 6

### Cessation of Food Intake and the Generation of Endogenous Energy from Fat are Some of the Physiological Hallmarks of an Animal in Deep Torpor

Methodology:

Animals: Wild-type (e.g., C57/B6), mPer1^{-/-}/mPer2^{m/m} and mPer1^{-/-}/mPer2^{m/m} female mice aged between about 8 and about 10 weeks were housed in a standard animal maintenance facility under a about 12 hours/about 12 hours light/dark cycle¹²⁻¹³. For about 12 hours/about 12 hours dark-dark or constant darkness studies, mice were placed inside a circadian chamber beginning at CT12 for about 48 hours under constant darkness before the mice were used for the indicated studies. All manipulations of constant darkness mice were carried out a under a red light of about 15 watts³⁰ and under institutionally approved animal protocol HSC-AWC 04-022.

Northern blot and RT-PCR analysis: Tissues were collected and frozen in liquid nitrogen and stored at about −80 C. The total RNA was isolated from mouse livers following standard procedures.³¹ Twenty micrograms of total RNA was separated by electrophoresis and transferred onto a nylon membrane. The blots were hybridized with ³²P-labeled cDNA probes, washed and exposed to X-ray film as previously described.³⁰ Colipase probe was the complete cDNA (e.g., Genbank No: BC042935); the Gapdh probe was the Pst I fragment of rat Gapdh cDNA.³² The primer pair used to measure colipase expression was SEQ ID NO:1 5′TTGTTCTTCTGCTTGTGTCCCT 3′ and SEQ ID NO:25′ AGTCGAGG CAGATGCCATAGTT 3′, The primer pair used to measure Gapdh expression as an internal control was SEQ ID NO:35′ AAGCCCATCACCATCTTCCA 3′ and SEQ ID NO:45′ ATGGCATGGACTGTGGTCAT 3′. A 720 bp probe for mouse pancreatic lipase related protein 2 (mPlrp2) was generated by RT-PCR using oligos LipaseF SEQ ID NO:55′-CGGTTGGACCCATCGGATGCCATG-3′ and LipasaeR SEQ ID NO:65′-GAACTCTTTCCCGTC TTTACCGCG-3′ from liver mRNA.

Hepatic colipase activity assay: Livers were removed from mice under ambient light (e.g., ZT0, ZT12) or under a red light of about 15 watts (e.g., CT0, CT12) and protein extracts were prepared as previously described.³³ The samples were heated for about 15 minutes at about 65 C to inactivate endogenous lipases. The protein content of the extracts was determined by the BCA method (Pierce). The heat-inactivated samples were assayed for the presence of colipase using the [3H] Triolein as substrate as previously outlined.³⁴

HPLC analysis for adenine nucleotides: Blood was rapidly removed from mice and frozen in liquid Nitrogen. Nucleotides were extracted from frozen samples using about 0.4 N perchloric acid as previously described.³⁵ Briefly, about 425 μl of ice-cold about 0.4 N perchloric acid was added to frozen blood samples and mixed. After about 10 μl was removed for protein determination, the remainder was centrifuged at about 14,000×g for about 10 minutes at about 4° C. The supernatant (e.g., 305 μl) was transferred to a clean tube, neutralized with about 178 μl of about 0.6 M KHCO₃/about 0.72 M KOH and acidified with about 55 μl of about 0.18 M ammonium phosphate solution (pH 5.1) and one drop of dilute phosphoric acid. The samples were centrifuged and the supernatants were stored at about −80° C. for analysis. Blood extracts and adenine nucleotides ATP, ADP, AMP, c-AMP and adenosine (e.g., from Sigma, Mo., USA) were separated and quantified using reversed-phase HPLC (e.g., Waters, Millipore Corp., Bedford, Mass.) analysis on a Partisphere bonded phase reverse phase C₈ cartridge column at a flow rate of 1.5 ml/minutes.³⁶ The mobile phase was 0.02 M N₄H₂PO₄, pH 5.1, with a superimposed methanol gradient: about 0% for about 0-4 minutes, about 0-8% for about 46 minutes, about 8-20% for about 6-8 minutes, and about 20% for about 8-18 minutes.

Injection of 5′-AMP, Adenosine, NECK and dipyridamole: The indicated dosage of 5′-AMP, adenosine, NECK and dipyridamole (e.g., from Sigma, Mo., USA) were administered into C57/B6 by intraperitoneal (IP) injection in light-dark cycle. After injection, mice were maintained for desired period length and then sacrificed. Total RNA from liver tissue was isolated and analyzed using northern blot¹⁹ and RT-PCR. Core body temperature (CBT) was measured at ambient room temperature (e.g., between about 23 and about 24° C.) before and after each injection with a rectal thermometer.

Metabolic Stress Studies: Measurement of core body temperature and AMP level in the blood during the fasting time course in constant darkness cycle was conducted with two groups of mice. Fed constant darkness mice were used as control group. The fasted constant darkness mice had their chow removed starting at CT2. Torpor was detected by CBT measurement and animals in torpor were either sacrificed for blood samples or given the food at the third CT2. Food and water intakes and body weight were measured at every ZT2 or CT2 for six continuous days in light-dark and constant dark cycles. Glucose and free fatty acid level in serum was measured by a glucose assay kit from BioAssay Systems (Hayward, Calif., USA) and a free fatty acid assay kit from Roche Applied Science (Penzberg, Germany).

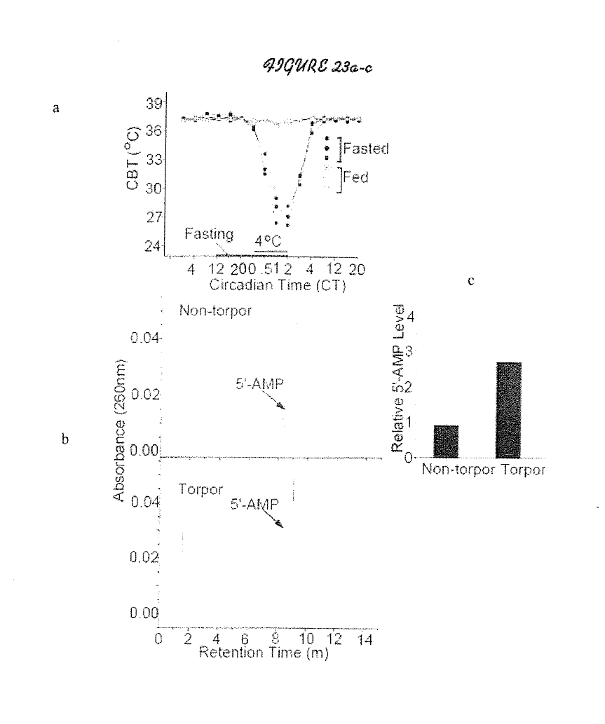

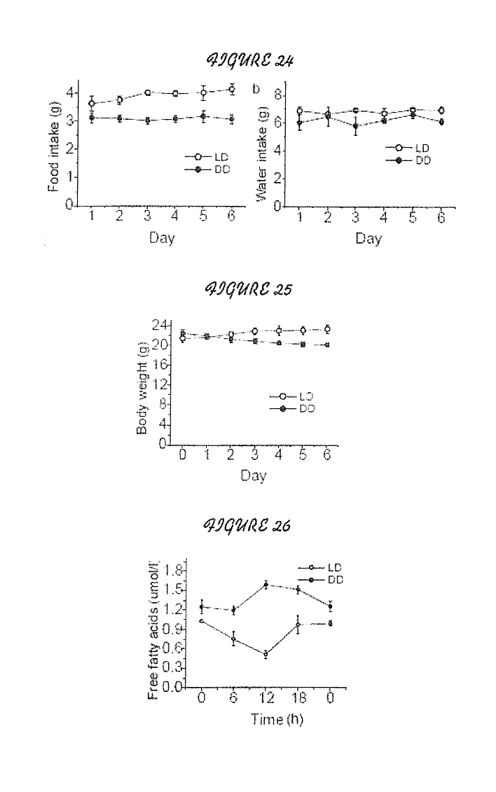

The targeted activation of procolipase by constant darkness is physiological since procolipase mRNA encodes for 2 peptides that are important for these biological events. The amino-terminal sequence of mClps is a penta-peptide (VPDPR) that is post-translationally cleaved from the colipase enzyme. This penta-peptide, known as enterostatin is a satiety regulator. For example, FIGS. 24a and 24b are graphs of consumption per day of food and water illustrating that constant darkness mice consumed less food and water than light-dark mice. This is consistent with previous observations of constant darkness versus light-dark cycle of rats.¹⁷

FIG. 25 is a graph of body weight per day that illustrates the body weight of constant darkness mice declined over the corresponding period studied. FIG. 26 is a graph of free fatty acids in the serum over time illustrating that free fatty acids in the serum of constant darkness mice were increased and is consistent with recent observations that large mammals kept in constant darkness have higher serum free fatty acids than those maintained in light-dark environment.¹⁸

Membrane-anchored and circadian-regulated ecto-5′-nucleotidase controls the extracellular level and mediates the intracellular action of 5′-AMP.^{14, 19, 20, 21} Northern blot analysis confirmed that expression of the ecto-5′-nucleotidase gene in light-dark mice is regulated in a circadian manner and is dampened in constant darkness animals (data not shown). Ecto-5′-nucleotidase dephosphorylates 5′-AMP to adenosine, which is taken into the cell by nucleoside transporters.²² Intracellular adenosine is primarily phosphorylated to 5′-AMP by adenosine kinase because its Kₘ for adenosine is about one or two orders of magnitude lower than that of adenosine deaminase.¹⁹ Mouse genetic studies have implicated the circadian clock in metabolic homeostasis.²³⁻²⁴ The regulatory actions of 5′-AMP on four allosteric enzymes involved in metabolism are well established. One such allosteric enzyme is the AMP-dependent protein kinase (AMPK) which is activated by 5′-AMP.²⁵ AICAR (5-aminoimidazole-4-carboxamide ribonucleoside) a 5′-AMP analog is known to increase fatty acid oxidation in rat muscle via AMPK.²⁶

In addition to AMPK, 5′-AMP is a positive and a negative regulator of the allosteric enzymes fructose 1,6-diphosphatase (FDP) and phosphofructokinase (PFK), respectively.²⁷ FDP is the rate-limiting enzyme for gluconeogenesis and it converts fructose 1,6-diphosphate to fructose 6-phosphate. FDP has 3 binding sites for 5′-AMP that inhibit its enzymatic activity thereby limiting gluconeogenesis. In the opposing direction, PFK is a rate-limiting enzyme for glycolysis. PFK converts fructose 6-phosphate into fructose 1,6-diphosphate, utilizing an ATP molecule. In contrast to FDP, the activity of PFK is enhanced by 5′-AMP thereby increasing the rate of glycolysis.

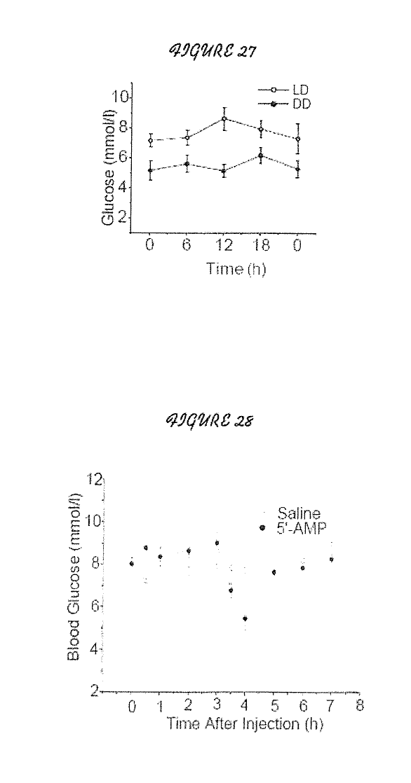

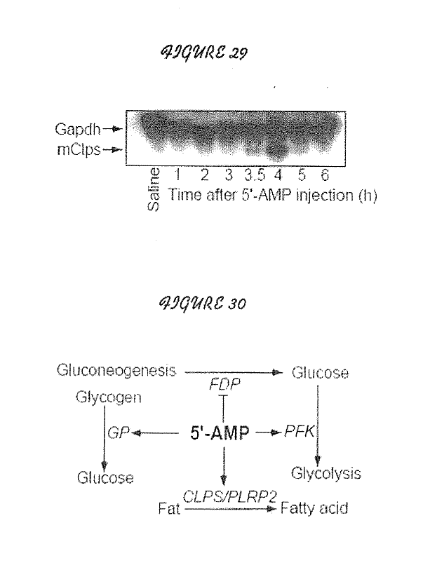

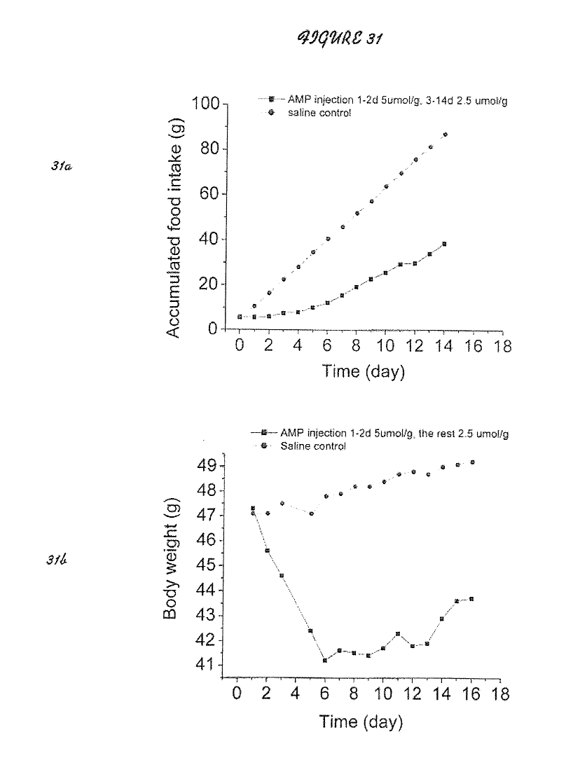

FIG. 27 is a graph of glucose concentration over time that illustrates, that in constant darkness mice where 5′-AMP is elevated, the blood glucose in constant darkness mice was significantly lower than light-dark mice and is consistent with previous studies in constant darkness versus light-dark rats.²⁸ FIG. 28 is a graph of the concentration of glucose in the blood over time and FIG. 29 is a Northern blot illustrating the 5′-AMP activation of mClps expression in light-dark mice is reciprocally linked to blood glucose levels. The activity of FDP is inhibited when 5′-AMP is injected into light-dark mice, thereby blocking gluconeogenesis. Conversely, 5′-AMP activates PFK to enhance the rate of glycolysis. Together, the positive and negative action of 5′-AMP on the activities of PFK and FDP, respectively, lead to a depletion of the blood glucose pool. The transient rise observed in blood glucose levels could be a result of a first level metabolic response to replenish this pool. The rate-limiting enzyme glycogen phosphorylase, which breaks down stored glycogen into glucose 1-phosphate, is another allosteric enzyme that is activated by 5′-AMP.²⁹ When stored glycogen depletion reaches a critical stage, blood glucose levels decline. To conserve glucose necessary for brain function (e.g., see FIG. 6 and FIG. 14), an alternative energy source for peripheral organs from fat catabolism is then activated monitored by the expression of mClps (e.g., see FIG. 4b).

FIG. 30 is a chart illustrating the role of 5′-AMP in metabolic signaling. 5′-AMP is a pivotal metabolic signal whose circulatory level determines the state of the body energy supply between glucose, glycogen and fat. The action of 5′-AMP and its analogs in humans may form a new class of therapeutic agents for human obesity and insulin-resistant type-2 diabetes. The ability of 5′-AMP to induce torpor is a useful tool in CBT management during major surgery or emergency trauma response from accident, combat and strokes. Additionally, in metabolic biochemistry the “futile cycle” burns up an ATP molecule between FDP and PFK activities.²⁷ However, the endogenous clock controls 5′-AMP levels and therefore the “futile cycle” is a circadian metabolic cycle.

In addition, daily injection of 5′-AMP into morbid obese mouse (O_{b}/O_{b}), which is deficient in leptin result in weight loss and lower satiety compared with O_{b}/O_{b} mouse injected with saline, as seen in FIGS. 31a and 31b. FIGS. 31a and 31b are graphs of the body weight and food intake after daily injection of 5′-AMP in O_{b}/O_{b} mice. During the first and second days, 5 umol/gbw of 5′-AMP was injected. This was then decrease to 2.5 umol/gbw for the rest of the studies. A gradual rise in food intake in the 5′-AMP injected mouse after 2 weeks suggest a new energy equilibrium.

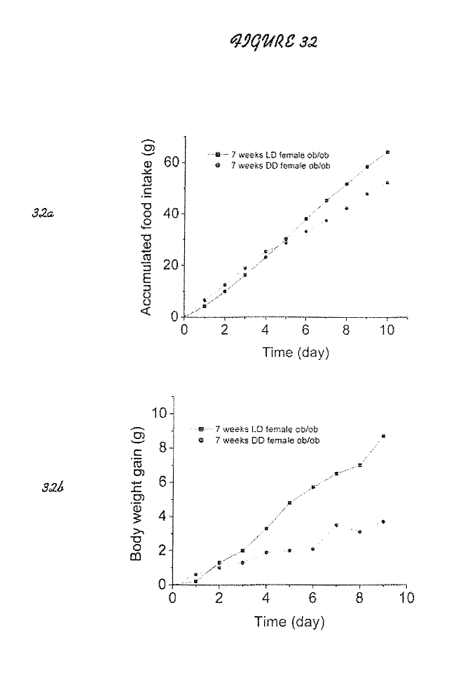

In addition, O_{b}/O_{b} mice kept in constant darkness also consumed less food and gain less weight that O_{b}/O_{b} mice kept in regular light-dark (12:12 hours) cycle, as seen in FIGS. 32a and 32b. FIGS. 32a and 32b are graphs of the effects of constant darkness on the satiety and body weight of O_{b}/O_{b} mice. Cumulative daily food consumption (grams) and weight gain (grams) over the corresponding period was monitored two 7 weeks old O_{b}/O_{b} female mice kept in typical light-dark (LD) and in constant darkness (DD). Note the decrease in total food consumption and rate of weight gain in the DD animal.

Furthermore, the present invention may be used for the treatment of cancer. Late stage and large tumors are highly hypoxic in nature since oxygen supply to tumor mass is limited. These tumors primarily generate its energy requirements through glycolytic processes widely known as the “Warburg hypothesis”. In contrast, normal cells utilized oxidative phosphorylation to generate the bulk of its ATP requirement. Studies have shown that the level of glycolytic enzymes such as PFK and FDP are highly elevated in many types and majority of human tumor. Mice kept in constant darkness have reverse metabolic parameters with respect to glucose and free fatty acids utilization compared to light-dark cycle mice. The present inventions have showed that level of blood glucose is lower but levels of free fatty acids are higher than light-dark cycle mice, mimicking those seen in hibernating mammals. This regulation of extracellular 5′-AMP level in constant darkness mice and light-dark cycle mice is correlated with the expression of the ecto-5′nucleotidase enzyme which degrades 5′-AMP into adenosine.

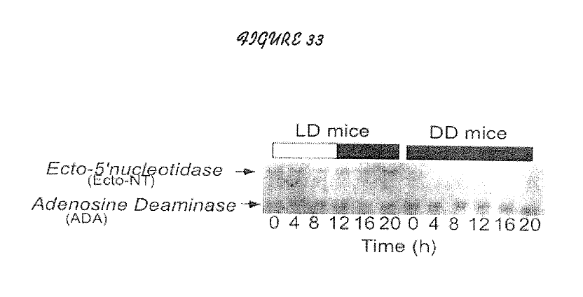

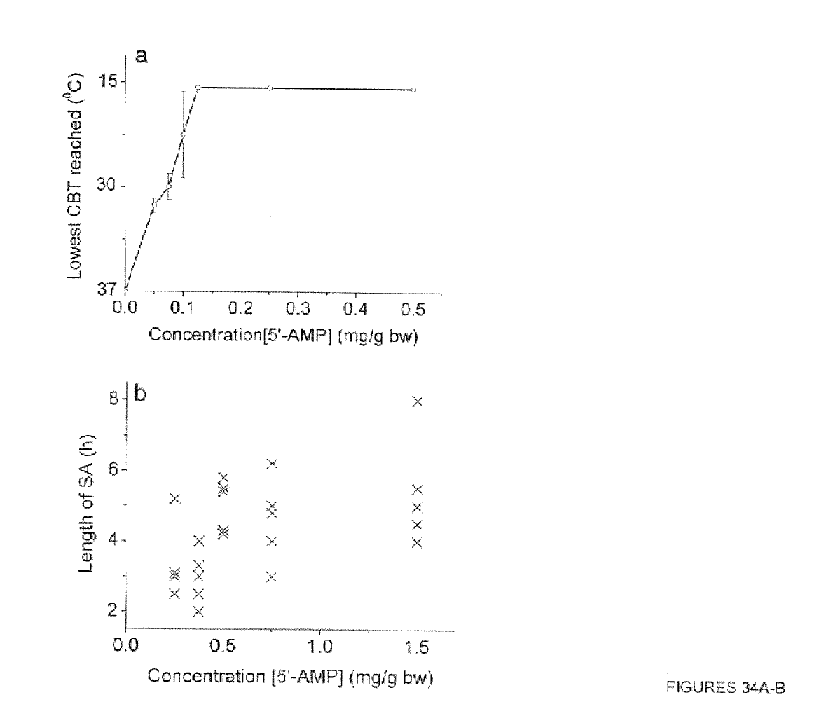

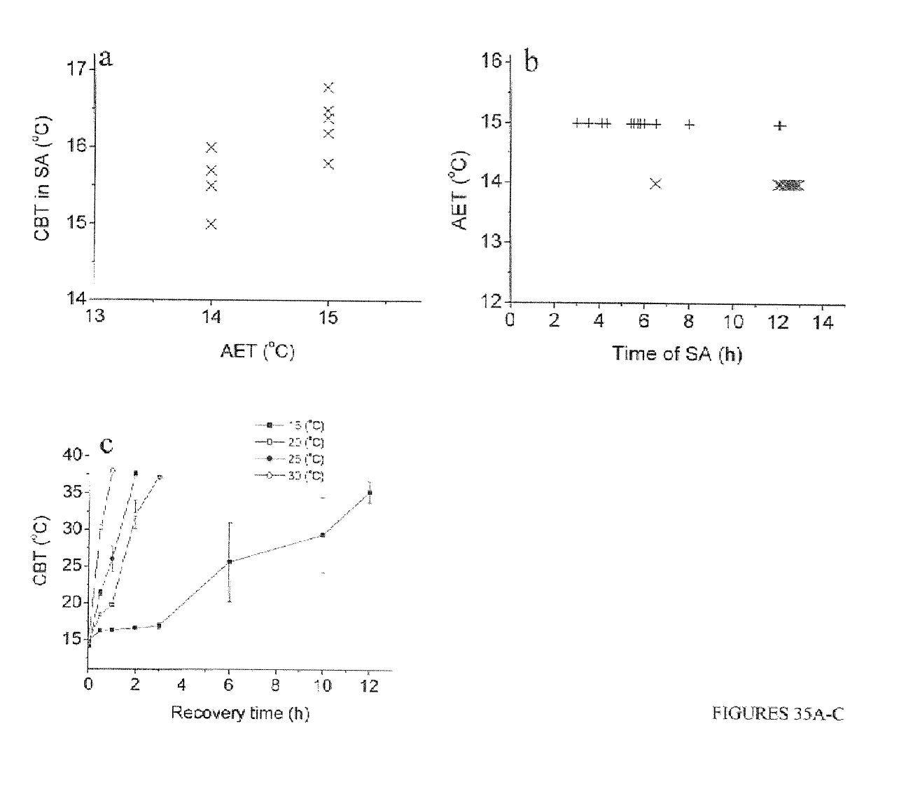

FIG. 33 is an image of a Northern blot illustrating the expression of ecto-5′nucleotidase gene is high in light-dark cycle mice but low in mice kept in constant darkness. Therefore, drugs that target the expression, activity or stability ecto-5′nucleotidase gene and enzyme would alter extracellular 5′-AMP levels in vivo. Furthermore, injections of 5′-AMP reduce the blood glucose levels, thus, putting patients in constant darkness or giving drugs that inhibit expression, activity or stability ecto-5′nucleotidase gene and enzyme and giving 5′-AMP will restrict the supply of glucose to tumor mass but enhanced the switch of normal cells to utilize fatty acids as energy source. Therefore, tumor cells that are unable to obtain adequate glucose will undergo necrosis and retard its growth thereby prolonging patient life span from the course of the disease.

### Example 7

### Controlled Suspended Animation of a Non-Hibernating Mammal

In the foregoing examples, it is shown that 5′-AMP mediates torpor and can induce a state of torpor in non-hibernating animals. In the present example, it is shown that 5′-AMP administration, in combination with a reduction in CBT, can be used to control and maintain a state of suspended animation (SA) in a non-hibernator, and exemplified through the use of laboratory mice. Mice entered SA when thermo-regulatory defenses were inhibited by 5′-AMP and CBT dropped below 17° C. The length of SA was sustained by the CBT that remains 1-2° C. above ambient environmental temperature (AET). Arousal from SA was spontaneous when AET was at least 15° C. and was inhibited below 14° C. Entry and arousal from SA were accompanied by distinct physiological responses and behaviors. Mice in SA were responsive to tactile stimuli, urinate and display sub-conscious behaviors. When fully aroused, the behavior of SA treated and untreated mice were indistinguishable. Our studies provide a basis for the conclusion that all mammals including humans can undergo reversible SA. Based on the foregoing studies, the inventors investigated the effects of low environmental temperature during caloric restriction on the mouse ability to enter SA.

Methods

Animals: The studies primarily used female mice (C57/B6), aged between 10 and 16 weeks. An identical response to 5′-AMP injection was also observed in male mice. Mice were housed in a standard animal facility under a 12-h/12-h light/dark cycle.

Experimental Procedures: Each mouse was injected intraperitoneally (IP) with the indicated dosages (e.g., 0.05-1.5 mg/g body weight) of 5′-AMP (Sigma catalog# A1752-25G) dissolved in phosphate buffered saline. They were immediately put into individual 500 ml pre-cooled beakers placed in a chamber at 4° C. Under these conditions, concentrations of 5′-AMP above 0.25 mg/gbw blocked all thermo-regulatory responses and allowed CBT to drop to 15±1.0° C. in 60±10 min. CBT of the mice was monitored by a digital thermometer (Fisher Scientific, USA, catalog 15-077-8) via a micro stainless steel probe (1 mm) placed 1 cm into the rectal opening of the mouse. Temperature readings were taken at 15-20 sec after insertion of the probe. Once the CBT dropped to 15±1.0° C., mice were then transferred to a regular mouse cage with bedding and kept in an environment chamber at a temperature set at 14±0.5° C. or 15±0.5° C. Mice in SA were visually monitored for arousal. Once arousal was apparent, the animals were returned to standard housing. After 12 h of SA, mice were returned to 20-22° C. AET housing with food and water given ad libitum. For 4 consecutive days after SA, food intakes were determined by weight differential of fresh chow and water after every 24 h at ZT2. Body weight was measured at every ZT2. Respiration rate was determined by placing a mouse inside an open-end 50 ml tube and the number of breaths were determined by counting the number of intakes and exhales over 10 sec intervals.

Results

When maintained at 4° C. AET, mice undergoing torpor at CT0 reduce their CBT to as low as 25° C. before spontaneously returning to a CBT of 37° C. These observations indicate caloric restriction-induced torpor combined with low AET did not generate SA in the mouse.

Next, the effects of low AET and 5′-AMP on mouse behavior was investigated. Mice injected with either saline or 5′-AMP were kept at 4° C. AET for about 60 min. Markedly different behavioral responses to 4° C. AET were observed. Thermo-regulatory defenses such as physical activity, raised hair follicles, curled up posture and shivering were observed in the saline injected animals. CBT measurement every 20 minutes showed minimal deviation from 37° C. In contrast, mice injected with 5′-AMP maintained a relaxed posture and no apparent shivering. CBT exhibited a rapid decline that was accompanied by distinct behavioral changes. As CBT dropped below 31° C., the mice retained a considerable cognitive response to tactile stimuli indicating a state of torpor (T). However, when CBT dropped to 18-22° C., the mice lost much of their locomotor mobility but had the ability to reverse flip (RF) to right themselves when they were placed on their backs or sides. When CBT dropped below 17° C., this RF ability was lost and the animals entered a state of SA. When laid on their sides or backs, the mice appeared in a suspended animation state with out-stretched limbs and low respiration rates. Notably, normal respiration rates of about 120 breaths per min were reduced by at least two-thirds in SA. Interestingly, mice in SA were responsive to tactile stimuli. When left alone, the mice would periodically displayed subconscious behaviors such as hind limb scratching of the lower body, flipping and rolling on their backs, yapping or yawning and urination.

Thus, even at CBT below 17° C., these behaviors indicate that the mice retained many neurological and physiological functions. If further cooled, mice were unable to survive long periods below 13° C. CBT. When the animals were maintained at 15° C. AET or higher, arousal from SA occurred spontaneously. Arousal was apparent when the mice regained the ability to perform a RF either spontaneously or if tactually stimulated. CBT measurements at arousal revealed a spontaneous rise above 17° C. This was followed shortly by a period of very intense shivering (S) that gradually lessened in intensity as the CBT rose. At CBT above 27° C., thermogenesis from shivering was not obvious and normal mouse behaviors such as self-grooming were apparent.

These observations raised questions concerning the relative contribution of 5′-AMP, AET and the CBT in mediating SA. To address these issues, the relationship between 5′-AMP and SA was investigated. First, the concentration dependence was determined of 5′-AMP in mediating a reduction in CBT to at least 15° C. when animals were kept at 4° C. AET for about 60 min. Under these conditions, the effective dosage (ED50) of 5′-AMP that induced at least 50% of the mice to enter SA was 0.125 mg/gbw (FIG. 34a).

Next, the concentration dependence of 5′-AMP on the length of SA was tested. Five groups of mice (n=5) were injected with 5′-AMP doses ranging from 0.25 to 1.5 mg/gbw, cooled at 4° C. until their CBT dropped below 17° C. and then transferred to a 15±0.5° C. AET to await spontaneous arousal. Surprisingly, there was considerable overlap in the arousal time between these non-linear concentrations of 5′-AMP (FIG. 34b). These observations suggest that the maintenance of SA was not directly driven by 5′-AMP concentration. Together with the behavioral response, the inventors propose that 5′-AMP primarily blocks the endogenous thermo-regulatory defenses that regulate CBT. Consistent with these observations, the 5′-AMP concentration curve for SA induction displayed a “set-point” or saturation response profile.

To identify the driver of SA, the relationship between CBT of mice and the AET was investigated. Once in SA, mice were maintained either at 14±0.5° C. or 15±0.5° C. AET. After 2 hours, measurement of CBT showed that the mice maintained a body temperature that was 1-2° C. above that of the AET (FIG. 35a). Next, the arousal responses of mice kept at 14±0.5° C. or 15±0.5° C. AET were compared. The majority of mice maintained at 15±0.5° C. AET could spontaneously aroused from SA (FIG. 35b). In contrast, the majority of mice maintained at 14±0.5° C. did not arouse from SA, even after 12 hours. When these mice were subsequently placed at a higher AET, they aroused spontaneously and could enter the S stage. A period of SA longer than 14 h was associated with a decline in the ability of the animal to enter the S stage after arousal by warming. Mice that failed to enter S stage died within 24 h. The underlying cause(s) of death remains unclear.

Together, the foregoing studies indicate that CBT is a major driver of SA in direct response to a low AET. To obtain additional support for this conclusion, the effects of AET on the recovery rate from SA was investigated. Groups of mice (n=4) in SA with a CBT of about 15° C. were transferred into environments with increasing AET's. The time required for each animal's CBT to return to 37° C. was measured (FIG. 35c). A direct correlation between the increase in AET and the recovery rate from SA was seen. To determine whether there were any long-term negative effects from SA, food intake and body weight were measured for several days as an indirect assessment of health status. Mice (n=4) that exited SA spontaneously were used for these studies. Indeed, a moderately lower food intake was observed during the first day after SA but no significant difference in body weight.

In summary, this example demonstrates the ability to initiate, maintain and terminate SA of a subject, as exemplified by the laboratory mouse.

### Example 8

### Blood Glucose Homeostasis and Adenylates Equilibrium in Suspended Animation

Animals. The studies primarily used female mice (C57/B16), aged between 10 and 16 weeks. An identical response to 5′-AMP injection was also observed in male mice. Mice were housed in a standard animal facility under a 12-h/12-h light/dark cycle.

Experimental Procedures. Each mouse was injected intraperitoneally (IP) with the appropriate dosages of 5′-AMP (Sigma catalog# A1752-25G) dissolved in phosphate buffered saline. They were immediately put into individual 500 ml pre-cooled beakers placed in a chamber at 4° C. Under these conditions, concentrations of 5′-AMP above 0.25 mg/gbw blocked all thermo-regulatory responses and allowed CBT to drop to 15±10.0° C. in 60±10 min. CBT of the mice was monitored by a digital thermometer (Fisher Scientific, USA, catalog 15-077-8) via a micro stainless steel probe (1 mm) placed 1 cm into the rectal opening of the mouse. Temperature readings were taken at 15-20 sec after insertion of the probe. Once the CBT dropped to 15±1.0° C., mice were then transferred to a regular mouse cage with bedding and kept in an environment chamber at a temperature set at 14±0.5° C. or 15±0.5° C. Accidental overcooling revealed that mice were unable to survive long period below 13° C. CBT. Mice in SA were visually monitored for arousal. Once arousal was apparent, the animals were returned to standard housing. After 12 h of SA, mice were returned to 20-22° C. AET housing with food and water given ad labitum. For 4 consecutive days after SA, food intakes were determined by weight differential of fresh chow every 24 h at ZT2. Body weight was measured at every ZT2. Respiration rate was determined by placing a mouse inside an open-end 50 ml tube and the number of breaths were determined by counting the number of intakes and exhales over 10 sec intervals. These studies were carried out under institutionally approved animal protocol HSC-AWC 04-022.

Blood Glucose Homeostasis in Suspended Animation

Observations that Siberian hamster undergoes torpor readily when given 2-deoxyglucose indicate that the biological process is link to glycolysis. That 5′-AMP is a key allosteric regulator of several rate-limiting enzymes involved in glucose homeostasis further implicates the importance of blood glucose

Analysis of serum glucose sample from mice at 2-3 h into SA was about 60% higher than control animals with CBT of 37° C. However, the serum blood glucose level drops to 40% below control animals at spontaneous arousal. Mice that were arouse by rewarming displays elevated blood glucose similar to those in SA (FIG. 37a). These observations suggest that spontaneous arousal unlike that achieved by rewarming was linked to a need to maintain blood glucose homeostasis. A decline in blood glucose level can be reversed by increasing gluconeogenesis and reduced glucose need of major organs through μ-oxidation of fatty acids. The activity of colipase and its enzymatic partners catabolized fat into fatty acids and its novel expression in major organs would implicate a transition from carbohydrates to fatty acids usage. Liver RNA obtained from mice in normal state, SA, arousal by rewarming and spontaneous arousal was analyzed for procolipase expression by Northern blot analysis. Only in mice that had aroused spontaneously was robust level of procolipase expression detected (FIG. 37b). Thus, low blood glucose level is associated with the activation of fat catabolism in the major organs.

Adenylates Equilibrium Role in Suspended Animation

To gather insight to the biochemical role played by the injected 5′-AMP in SA, the level of adenylates and its catabolic products was analyzed in blood, liver and muscle of mice in control animals with CBT of 37° C., SA and at spontaneous arousal. The HPLC analysis of the control animals revealed that the normal adenylates levels were not the same in these three organs, with liver and blood showing much higher ATP level than in muscle. (FIGS. 38 a through d) However, only during SA did the relative AMP to ATP ratio increases by about 5 fold in the blood but not in liver or muscle (FIG. 39a). There was little or no change of adenylate ratio in liver and muscle in all three behaviors state. A steep rise in 5′-AMP and a moderate drop in ATP level account for the increased AMP/ATP ratio during SA in blood. At spontaneous arousal, the elevated AMP/ATP ratio returns to normal (FIGS. 39a-c). The ADP to ATP ratios remains relatively constant in all tissues and behavior states (data not shown). The presence of purine catabolic products including inosine, hypoxanthine and uric acid in blood during SA further implicates its uptake and catabolism of the injected 5′-AMP (FIGS. 38c and 38d). HPLC analysis further showed that mice in SA given a second injection of 5′-AMP displayed a steep drop in ATP level (FIG. 38d). Such low ATP level maybe inadequate for red blood cells function and could be a likely the reason for the observed fatality effect. Interestingly, the level of 5′-AMP is maintained at a threshold level and its excess was rapidly catabolized as indicated by a large increase of inosine level. This finding is consistent with the observation that excess 5′-AMP do not lengthen SA. Together, these observations indicate that the adenylates equilibrium (ATP+5′-AMP< >2ADP) regulates the level of ATP and 5′-AMP even during metabolic unfavorable conditions.

It will be understood that particular embodiments described herein are shown by way of illustration and not as limitations of the invention. The principal features of this invention can be employed in various embodiments without departing firm the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Heldmaier, G., Ortmann, S., and Elvert, R., Natural hypometabolism during hibernation and daily torpor in mammals. Respir Physiol Neurobiol. 141, 317-329 (2004).
2. Perret, M., and Aujard, F., Daily hypothermia and torpor in a tropical primate: synchronization by 24-h light-dark cycle. Am J Physiol Regul Integr Comp Physiol. 281, 1925-1933 (2001).
3. Heller, H. C., and Ruby, N. F., Sleep and circadian rhythms in mammalian torpor. Annu Rev Physiol. 66, 275-289 (2004).
4. Wassmer, T., and Wollnik, F. Timing of torpor bouts during hibernation in European hamsters (Cricetus cricetus L.). J Comp Physiol [B]. 167, 270-279 (1997).
5. Morgan, P. J., Ross, A. W., Mercer, J. G., and Barrett, P., Photoperiodic programming of body weight through the neuroendocrine hypothalamus. J Endocrinol. 177, 27-34 (2003).
6. Ruby, N. F., Dark, J., Burns, D. E., Heller, H. C., and Zucker, I., The suprachiasmatic nucleus is essential for circadian body temperature rhythms in hibernating ground squirrels. J Neurosci. 22, 357-364 (2002).
7. Gavrilova, O., et al., Torpor in mice is induced by both leptin-dependent and -independent mechanisms. Proc Natl Acad Sci USA. 96, 14623-14628. (1999).
8. Overton, J. M., and Williams, T. D., Behavioral and physiologic responses to caloric restriction in mice. Physiol Behav. 81, 749-754 (2004).
9. Ruby, N. F., Dark, J., Heller, H. C., and Zucker, I., Ablation of suprachiasmatic nucleus alters timing of hibernation in ground squirrels. Proc Natl Acad Sci USA. 93, 9864-9868 (1996).
10. Lowe, M. E., Molecular mechanisms of rat and human pancreatic triglyceride lipases. J Nutr. 127, 549-557 (1997).
11. Lowe, M. E., Rosenblum, J. L., McEwen, P., and Strauss, A. W., Cloning and characterization of the human colipase cDNA. Biochemistry. 29, 823-828 (1990).
12. Zheng, B., et al., Nonredundant roles of the mPer1 and mPer2 genes in the mammalian circadian clock. Cell. 105, 683-694 (2001).
13. Zheng, B., et al., The mPer2 gene encodes a functional component of the mammalian circadian clock. Nature. 400, 169-173 (1999).
14. Thompson, L. F., Ruedi, J. M., Glass, A., Low, M. G., and Lucas, A. H., Antibodies to 5′-nucleotidase (CD73), a glycosyl-phosphatidylinositol-anchored protein, cause human peripheral blood T cells to proliferate. J Immunol. 143, 1815-1821 (1989).
15. Thorn, J. A., and Jarvis, S. M., Adenosine transporters. Gen Pharmacol. 27, 613-620 (1996).
16. Erlanson-Albertsson, C., and Larsson, A., The activation peptide of pancreatic procolipase decreases food intake in rats. Regul Pept. 22, 325-331 (1988).
17. Stoynev, A. G., and Ikonomov, O. C., Effect of constant light and darkness on the circadian rhythms in rats: 1. Food and water intake, urine output and electrolyte excretion. Acta Physiol Pharmacol Bulg. 9, 58-64 (1983).
18. Alila-Johansson, A., Eriksson, L., Soveri, T., and Laakso, M. L., Daily and annual variations of free fatty acid, glycerol and leptin plasma concentrations in goats (Capra hircus) under different photoperiods. Comp Biochem Physiol A Mol Integr Physiol. 138, 119-131 (2004).
19. Arch, J. R. and Newsholme, E. A., Activities and some properties of 5′-nucleotidase, adenosine kinase and adenosine deaminase in tissues from vertebrates and invertebrates in relation to the control of the concentration and the physiological role of adenosine. Biochem. J. 174, 965-977 (1978).
20. Schnebli, H. P., Hill, D. L., and Bennett, L. L. Jr., Purification and properties of adenosine kinase from human tumor cells of type H. Ep. No. 2. J Biol Chem. 242, 1997-2004 (1967).
21. von Mayersbach, H. and Klaushofer, K., Circadian variations of 5′-nucleotidase activity in rat liver. Cellular and Molecular Biology, Including Cyto-enzymology 24, 73-79 (1979).
22. Thorn, J. A. and Jarvis, S. M., Adenosine transporters. Gen. Pharmacol. 27, 613-620 (1996).
23. Rudic, R. D., et al., BMAL1 and CLOCK, two essential components of the circadian clock, are involved in glucose homeostasis. PLoS Biol. 2, e377 (2004).
24. Turek, F. W., et al., Obesity and metabolic syndrome in circadian Clock mutant mice. Science. 308, 1043-1045 (2005).
25. Lindsley, J. E., and Rutter, J., Nutrient sensing and metabolic decisions. Comp Biochem Physiol B Biochem Mol Biol. 139, 543-559 (2004).
26. Kaushik, V. K., et al., Regulation of fatty acid oxidation and glucose metabolism in rat soleus muscle: effects of AICAR. Am J Physiol Endocrinol Metab. 281, 335-340 (2001).
27. Lehninger, A. L., Biochemistry: The biosynthesis of carbohydrates, pp 623-657. 2^{nd} eds, Worth Publishers, Inc Park Avenue South, NY (1977).
28. Ahlersova, E., Ahlers, I., Toropila, M., and Smajda, B., Influence of light regimens on circadian changes in the blood glucose and tissue glycogen concentration in the rat. Physiol Bohemoslov. 31, 57-64 (1982).
29. Garcia-Fuentes, L., Camara-Artigas, A., Lopez-Mayorga, O., and Baron, C., Thermodynamic characterization of 5′-AMP binding to bovine liver glycogen phosphorylase a. J Biol Chem. 271, 27569-27574 (1996).
30. Kaasik, K., and Lee, C. C., Reciprocal regulation of haem biosynthesis and the circadian clock in mammals. Nature. 430, 467-471 (2004).
31. Chirgwin, J. M., Przybyla, A. E., MacDonald, R. J., and Rutter, W. J. Isolation of biologically active ribonucleic acid from sources enriched in ribonuclease. Biochemistry. 18, 5294-5299 (1979).
32. Fort. P., et al., Various rat adult tissues express only one major mRNA species from the glyceraldehyde-3-phosphate-dehydrogenase multigenic family. Nucleic Acids Res. 13, 1431-1442 (1985).
33. D'Agostino, et al., Decreased postnatal survival and altered body weight regulation in procolipase deficient mice. J. Biol. Chem. 277:7170-7177 (2002).
34. Lowe, M. E., in Methods in Molecular Biology: Lipase and Phospholipase Protocols (Doolittle, M. H. and Reue, K., eds), pp. 59-70, Humana Press Inc., Totowa, N.J. (1998).
35. Blackburn, M. R., Datta, S. K., and Kellems, R. E., 1998. Adenosine deaminase-deficient mice generated using a two-stage genetic engineering strategy exhibit a combined immunodeficiency. J. Biol. Chem. 273:5093-5100.
36. Knudsen, T. B. et al., Effects of (R)-deoxycoformycin (pentostatin) on intrauterine nucleoside catabolism and embryo viability in the pregnant mouse. Teratology. 45:91-103 (1992).

What is claimed is:

1. A method of inducing a state of hypothermia, torpor, or suspended animation in a subject comprising administering an amount of 5′-AMP effective to induce the state of torpor or suspended animation.

2. The method of claim 1, further comprising determining that the subject is in a state of torpor or suspended animation.

3. The method of claim 1, further comprising subjecting the subject to an ambient environmental temperature that is below about 30° C. after administration of the 5′-AMP.

4. The method of claim 3, wherein the subject is subjected to an ambient environmental temperature that is between about 25° C. and about 1° C.

5. The method of claim 4, wherein the subject is subjected to an ambient environmental temperature that is between about 20° C. and about 4° C.

6. The method of claim 1, further comprising lowering the core body temperature of the subject to less than about 37° C.

7. The method of claim 1, further comprising lowering the core body temperature of the subject to about 32° C. or less.

8. The method of claim 6, wherein the core body temperature of the subject is lowered to between about 15° C. and about 20° C.

9. The method of claim 8, wherein the core body temperature of the subject is lowered to between about 13° C. and about 15° C.

10. The method of claim 1, wherein the torpor is deep torpor.

11. The method of claim 1, wherein the 5′-AMP is administered by subcutaneous injection, intramuscular injection, intravenous injection, intraperitoneal injection, nasal administration, intravaginal administration, intranasal administration, intrabronchial administration, intraocular administration, intraaural administration, intracranial administration, oral consumption, parenteral administration, rectal administration, sublingual administration, topical administration, transdermal administration or combination thereof.

12. The method of claim 1, wherein the 5′-AMP is administered in the form of a capsule, caplet, softgel, gelcap, suppository, film, granule, gum, pastille, pellet, chewable tablet, troche, lozenge, disk, poultice, wafer, creams, lotions, ointments, aerosol sprays, roll-on liquids, roll-on sticks, transdermal patches, subcutaneous implants, pads or combinations thereof.

13. The method of claim 12, wherein the 5′-AMP is disposed for extended release in a biodegradable carrier.

14. The method of claim 1, wherein the 5-AMP is administered in a pharmaceutically acceptable dosage form that further comprises a pharmaceutically acceptable carrier.

15. The method of claim 14, wherein the pharmaceutically acceptable carrier comprises an aerosol propellant selected from nitrogen, carbon dioxide, propane, butane, isobutene, pentane, isopropane, fluorocarbons, dimethylether and mixtures thereof.

16. The method of claim 1, wherein the 5′-AMP is administered in a dosage form that further comprises at least one agent selected from the group consisting of emollients, water, inorganic powders, foaming agents, emulsifiers, fatty alcohols, fatty acids and combinations thereof.

17. The method of claim 1, wherein the subject is a human.

18. The method of claim 1, wherein the subject is a laboratory animal.

19. The method of claim 18, wherein the laboratory animal is a mouse.

20. The method of claim 1, wherein the effective amount of 5′-AMP ranges from about 5 mg/kg to about 7.5 gm/kg body weight.

21. The method of claim 1, wherein the effective amount of 5′-AMP ranges from about 15 mg/kg to about 1.5 gm/kg body weight.

22. The method of claim 21, wherein the effective amount ranges from about 25 mg/kg to about 250 mg/kg body weight.

23. The method of claim 20, wherein the effective amount ranges from about 5 mg/kg to about 15 mg/kg.

24. The method of claim 23, wherein the method further comprises at least partially submerging the subject in a water bath, wherein the temperature of the water bath is below about 32° C.

25. The method of claim 23, wherein the method further comprises at least partially covering the patient with a cooling blanket.

26. The method of claim 1, wherein the subject is suffering from a disease state to be treated by induction of a state of torpor or suspended animation.

27. The method of claim 26, wherein the disease state is a state of shock, trauma, a blood coagulation disorder, a side effect of chemotherapy, poisoning, a cardiac arrhythmia, hypothermia, burns, suffocation, inhalation injury, ventilation insufficiency, sepsis, anxiety, insulin shock, an infectious disease, cancer, carcinoma, near drowning, heart attack, congestive heart failure, decompression sickness, asthma, starvation, stroke, severe trauma, a head trauma, a brain trauma, a cerebrovascular injury, a cerebrovascular trauma, a neurological trauma, a neurological injury, a fever, a heatstroke, an eating disorder, anxiety, a seizure, epilepsy, insomnia or a sleeping disorder, diabetes, obesity, hypertension, hyperthyroidism, hypothyroidism or combinations thereof.

28. The method of claim 1, wherein the subject is a transplant recipient, a transplant donor, in need of appetite suppression, a pre-surgical patient, a post-surgical patient, or a patient who has received or will be receiving a chemotherapeutic.

29. The method of claim 1, wherein the amount of 5′-AMP is effective to reduce the core body temperature, reduce the external body temperature, reduce the metabolic rate, reduce the heart rate and combinations thereof.

30. The method of claim 1, further comprising administering to the subject a second pharmaceutical agent.

31. The method of claim 30, wherein the second pharmaceutical agent comprises an adjunctive agent, an anticoagulant, an inotropic agent a chronotropic agent, an analgesic agent, an anesthetic agent, a neuroprotective agent, an antiarrhythmic agent, or a calcium channel blocker.

32. A method of reducing blood glucose level in a subject comprising administering to the subject an amount of 5′-AMP effective to reduce blood glucose levels in the individual.

33. The method of claim 32, wherein the subject is suffering from diabetes, obesity or is in need of appetite suppression.

34. A method of modifying the metabolic state of a tissue to increase fatty acid metabolism in the tissue relative to glycolysis therein, comprising administering to the subject an amount of 5′-AMP effective to increase fatty acid metabolism.

35. The method of claim 34, wherein the tissue is comprised in a subject.

36. The method of claim 34, wherein the subject is a transplant recipient, a transplant donor, a pre-surgical patient, a post-surgical patient, or a patient who has received or will be receiving a chemotherapeutic.

37. The method of claim 34, wherein the tissue has been removed from a subject.

38. The method of claim 37, wherein the tissue comprises part or all of an organ.

39. The method of claim 38, wherein the organ is a transplant organ.

40. The method of claim 34, further comprising determining that the fatty acid metabolism in the subject has been increased.

41. The method of claim 34, wherein the patient is suffering from diabetes, obesity or is in need of appetite suppression.

42. The method of claim 34, wherein the tissue is a solid tumor.

43. A method of reducing the core body temperature of a subject comprising administering to the subject an amount of 5′-AMP that is effective to reduce the subject's core body temperature.

44. The method of claim 43, further comprising determining the subject's core body temperature.

45. The method of claim 43, wherein the subject is in a state of shock, or has a trauma, a blood coagulation disorder, side effects of chemotherapy, poisoning, a cardiac arrhythmia, hypothermia, burns, suffocation, inhalation injury, ventilation insufficiency, sepsis, anxiety, insulin shock, an infectious disease, cancer, carcinoma, near drowning, heart attack, congestive heart failure, decompression sickness, asthma, starvation, stroke, severe trauma, a head trauma, a brain trauma, a cerebrovascular injury, a cerebrovascular trauma, a neurological trauma, a neurological injury, a fever, a heatstroke, an eating disorder, anxiety, a seizure, epilepsy, insomnia and sleeping disorders, diabetes, obesity, hypertension, hyperthyroidism, hypothyroidism, is to undergo a surgical procedure, is a transplant patient, is in need of appetite suppression, is a patient who has received or will be receiving a chemotherapeutic, or combinations thereof.

46. A method of reducing the metabolic rate of a subject comprising administering to the subject an amount of 5′-AMP that is effective to reduce the subject's metabolic rate.

47. The method of claim 46, further comprising assessing the subject's metabolic rate.

48. The method of claim 46, wherein the subject is in a state of shock, have a trauma, a blood coagulation disorder, side effects of chemotherapy, poisoning, a cardiac arrhythmia, hypothermia, burns, suffocation, an inhalation injury, ventilation insufficiency, sepsis, anxiety, insulin shock, an infectious disease, cancer, carcinoma, near drowning, heart attack, congestive heart failure, decompression sickness, asthma, starvation, in need of appetite suppression, stroke, severe trauma, a head trauma, a brain trauma, a cerebrovascular injury, a cerebrovascular trauma, a neurological trauma, a neurological injury, a fever, a heatstroke, an eating disorder, anxiety, a seizure, epilepsy, insomnia and sleeping disorders, diabetes, obesity, hypertension, hyperthyroidism, hypothyroidism, is to undergo a surgical procedure, is a transplant patient, is a patient who has received or will be receiving a chemotherapeutic, or combinations thereof.

49. A pharmaceutical composition comprising a pharmaceutically effective amount of 5′-AMP sufficient to produce a state of torpor or suspended animation, a reduction in the core body temperature, an inotropic effect on the heart, a decrease in cell growth, a reduction in metabolic rate, a reduction in the blood glucose levels or combinations thereof.

50. The pharmaceutical composition of claim 49, wherein the composition is formulated for administration by subcutaneous injection, intramuscular injection, intravenous injection, intraperitoneal injection, nasal administration, intravaginal administration, intranasal administration, intrabronchial administration, intraocular administration, intraaural administration, intracranial administration, oral consumption, parenteral administration, rectal administration, sublingual administration, topical administration, transdermal administration or combination thereof.

51. The pharmaceutical composition of claim 49, wherein the 5′-AMP is formulated in the form of a capsule, caplet, softgel, gelcap, suppository, film, granule, gum, insert, a chewable tablet, a pastille, pellet, troche, lozenge, disk, poultice, wafer, a cream, a lotion, an ointment, an aerosol spray, a roll-on liquid, a roll-on stick, a transdermal patch, a subcutaneous implant, a pad, or is disposed for extended release in a biodegradable carrier.

52. The pharmaceutical composition of claim 49, further comprising a pharmaceutically acceptable carrier.

53. The pharmaceutical composition of claim 52, wherein the pharmaceutically acceptable carrier comprises an aerosol propellant selected from nitrogen, carbon dioxide, propane, butane, isobutene, pentane, isopropane, fluorocarbons, dimethylether and mixtures thereof.

54. The pharmaceutical composition of claim 52, wherein the pharmaceutically acceptable carrier comprises at least one agent selected from the group consisting of emollients, water, inorganic powders, foaming agents, emulsifiers, fatty alcohols, fatty acids and combinations thereof.

55. The pharmaceutical composition of claim 49, formulated in an injectable dosage form.

56. The pharmaceutical composition of claim 49, wherein the 5′-AMP is contained in a metered dosage in a vial or ampoule.

57. The pharmaceutical composition of claim 56, wherein the 5′-AMP is dispersed in an aqueous solution.

58. The pharmaceutical composition of claim 57, further comprising one or more pharmaceutical additives.

59. The pharmaceutical composition of claim 58, wherein the additives includes one or more buffers or physiologic salts.

60. The pharmaceutical composition of claim 56, wherein the vial or ampoule comprises a septum.

61. The pharmaceutical composition of claim 56, wherein dosage is metered to provide from 1 to 5 doses.

62. The pharmaceutical composition of claim 61, wherein each dose comprises from 1 to 500 gm of 5′-AMP.

63. The pharmaceutical composition of claim 62, wherein each dose comprises from 2 to 20 gm of 5′-AMP.

64. The pharmaceutical composition of claim 61 wherein each dose comprises an amount of 5′-AMP effective to deliver from 15 mg/kg to 7.5 gm/kg body weight to a subject.

65. The pharmaceutical composition of claim 61 wherein each dose comprises an amount of 5′-AMP effective to deliver from 25 mg/kg to 250 mg/kg body weight to a subject.

66. The pharmaceutical composition of claim 49, formulated and placed into a projectile for inducing a state of torpor or suspended animation in a subject.

67. The pharmaceutical composition of claim 49, further defined as an aerosol composition adapted for inducing torpor in a subject comprising a pharmaceutically effective amount of 5′-AMP and a propellant.

68. The pharmaceutical composition of claim 67, wherein the aerosol composition is in the form of an inhaler.

69. The pharmaceutical composition of claim 30, further comprising a second active agent.

70. The method of claim 69, wherein the second active agent is an adjunctive agent, an anticoagulant, an inotropic agent, a chronotropic agent, an analgesic agent, an anesthetic agent, a neuroprotective agent, an antiarrhythmic agent, or a calcium channel blocker.

71. A method of altering metabolic activity in a subject comprising administering a pharmaceutically effective amount of 5′-AMP to a subject, wherein the 5′-AMP alters the activity of one or more metabolic enzymes selected from procolipase, pancreatic lipase, pancreatic lipase related protein, phosphofructose kinase, fructose 1,6 diphosphatase, glycogen phosphorylase, and combinations thereof.
